(19) United States
(12) Patent Application Publication
(10) Pub. No.: US 2019/0330328 A1
(43) Pub. Date: Oct. 31, 2019

(54) METHODS OF TREATING NON-RADIOGRAPHIC AXIAL SPONDYLOARTHRITIS USING INTERLEUKIN-17 (IL-17) ANTAGONISTS

(71) Applicants: Christian Mann, Erlangen (DE); Brian Porter, Chester, NJ (US); Hanno Richards, Therwil (CH)

(72) Inventors: Christian Mann, Erlangen (DE); Brian Porter, Chester, NJ (US); Hanno Richards, Therwil (CH)

(21) Appl. No.: 15/766,043

(22) PCT Filed: Oct. 14, 2016
(86) PCT No.: PCT/IB2016/056166

§ 371 Date: Apr. 5, 2018

Related U.S. Application Data
(60) Provisional application No. 62/243,381, filed on Oct. 19, 2015.

(51) Int. Cl. C07K 16/24; A61K 39/395; A61P 19/02

(52) U.S. Cl. CPC C07K 16/244; A61K 2039/545; A61P 19/02; A61K 39/3955

(57) ABSTRACT

The present disclosure relates to methods for treating non-radiographic axial spondyloarthritis (nr-axSpA) patients and inhibiting the progression of structural damage in these patients, using Interleukin-17 (IL-17) antagonists, e.g., secukinumab. Also disclosed herein are uses of IL-17 antagonists, e.g., IL-17 antibodies, such as secukinumab, for treating nr-axSpA patients and inhibiting the progression of structural damage in these patients, as well as medicaments, dosing regimens, pharmaceutical formulations, dosage forms, and kits for use in the disclosed uses and methods.

### RELATED APPLICATIONS

The present application claims priority to U.S. Provisional Application No. 62/243,381, filed Oct. 19, 2015, the content of which is incorporated by reference herein in its entirety.

### TECHNICAL FIELD

The present disclosure relates to methods for treating non-radiographic axial spondyloarthritis (nr-axSpA) patients and inhibiting the progression of structural joint damage in these patients, using IL-17 antagonists, e.g., secukinumab.

### BACKGROUND OF THE DISCLOSURE

Axial Spondyloarthritis (axSpA) is a group of rheumatic disorders with spinal inflammation and inflammatory back pain as a common denominator. Patients with chronic back pain (onset before 45 years of age) are classified according to the Assessment of Spondyloarthritis international Society (ASAS) classification criteria (Rudwaleit et al 2009, Ann Rheum Dis; 68:770-76) for axSpA if they fulfill either the clinical arm or the imaging arm of the criteria. Based on the presence or absence of sacroiliitis on conventional X-ray radiographs, axSpA patients are sub-grouped into non-radiographic axSpA (nr-axSpA) and ankylosing spondylitis (AS). Patients with evidence for sacroiliitis on X-ray fulfilling the 1984 modified New York diagnostic criteria (van der Linden et al 1984, Arthritis Rheum; 27:361-8) are classified as having AS whereas patients who do not show sacroiliitis on X-ray but may show evidence of sacroiliitis on MRI are classified as having nr-axSpA.

The 2009 ASAS classification criteria for axSpA were introduced to establish standards that apply to patients with or without radiographic sacroiliitis by including both X-ray and MRI as imaging modalities. The diagnosis of nr-axSpA based on imaging can achieve up to 88% specificity and 67% sensitivity, whilst diagnosis based only on clinical parameters can achieve approximately 83% specificity and 570/% sensitivity (Sieper and van der Heijde 2013, Arthritis Rheum; 65:543-5 1). In addition to the differential identification of AS and nr-axSpA patients, the ASAS criteria allow for the implementation of clinical trials in the treatment of nr-axSpA, a disease entity for which there is an unmet medical need, with no approved therapies in the United States (Sieper 2012, Nat Rev Rheumatol; 8:280-87).

Studies and registry data have shown that nr-axSpA patients have similar levels of disease activity, pain, and health-related quality of life impairment as do AS patients (Wallis et al 2013, J Rheumatol; 40:2038-41). Commonality of etiopathogenic characteristics and the natural history of AS and nr-axSpA are the subjects of ongoing research. Disease parameters and response rates to treatment with tumor necrosis factor (TNF) antagonists are similar in patients with nr-axSpA and AS, supporting the concept that axSpA is one common disease with distinct stages (Song et al 2013, Ann Rheum Dis; 72:823-25). Progression from nr-axSpA to AS was observed in about 12% of nr-axSpA patients over the course of 2 years (Poddubnyy et al 2011, Ann Rheum Dis; 70:1369-74). However, it is estimated that 10-15% of nr-axSpA patients do not develop radiographic sacroiliitis on x-rays (Sieper and van der Heijde 2013, Arthritis Rheum; 65:543-51).

Non-steroidal anti-inflammatory drugs (NSAIDs) are considered first-line therapy for all patients with axSpA. Traditional disease-modifying antirheumatic drugs (DMARDs) such as methotrexate and sulfasalazine are not effective in the treatment of axSpA. Anti-TNF agents are approved therapies for patients with AS who continue to have active disease despite NSAIDs. In Europe, several anti-TNF agents are also approved for nr-axSpA. However, more than 60% of nr-axSpA patients treated with adalimumab or etanercept did not achieve an ASAS40 response in randomized clinical trials (Sieper et al 2013, Ann Rheum Dis; 72:815-22; Dougados et al 2014, Arthritis Rheum; 66:2091-2102). Moreover, TNF blockade does not result in long-term remission in axSpA, and responders usually relapse within a few weeks after interruption of treatment (Baraliakos et al 2005, Arthritis Res Ther; 7: R439-R444). While effective in treating the inflammatory symptoms. TNF antagonists do not prevent structural damage of the joints in axSpA which was primarily studied in AS (van der Heijde et al 2008a, Arthritis Rheum; 58:3063-70; van der Heijde et al 2008b, Arthritis Rheum; 58:1324-31).

### SUMMARY OF THE DISCLOSURE

Secukinumab (AIN457) is a high-affinity recombinant, fully human monoclonal anti-human interleukin-17A antibody of the IgG1/κ-class. Secukinumab binds to human IL-17A and neutralizes the bioactivity of this cytokine. IL-17A is the central lymphokine of a newly defined subset of inflammatory T cells (Th17) which appear to be pivotal in several autoimmune and inflammatory processes in some animal models. IL-17A is mainly produced by memory CD4+ and CD8+T lymphocytes and is being recognized as one of the principal pro-inflammatory cytokines in immune mediated inflammatory diseases.

A radiographic axSpA (ankylosing spondylitis; AS) Phase III study of secukinumab (150 mg SC at Weeks 0, 1, 2, and 3, followed by the same dose every 4 weeks) showed an ASAS40 response rate of 36.1% at Week 16. A clinically meaningful difference in ASAS40 response between the secukinumab 150 mg group and placebo was evident as early as at Week 1. In the Phase II study in AS, MRI imaging performed at baseline and at Weeks 6 and 28 showed a reduction of inflammation after 6 weeks which was maintained up to Week 28. Early improvements were especially noted in patients with higher baseline MRI scores (Baraliakos et al 2011, Arthritis Rheum. 63(Suppl 10):2486D).

Currently, there are no FDA-approved therapies available in the United States for nr-axSpA. Given the potential role of Th17 cells in the inflammatory infiltrate in spondyloarthritis, the activity of inflammation in early disease stages (such as nr-axSpA), the comparability of secukinumab to the TNF-alpha inhibitors certolizumab and etanercept in treating AS, and the early reduction in inflammation evidenced by MRI during AS trials of secukinumab, the long-term structural changes in axial joints may be amenable to modulation via IL-17 antagonism.

Accordingly, disclosed herein are methods of treating a patient having non-radiographic axial spondyloarthritis (nr-axSpA), comprising administering an IL-17 antagonist to a patient in need thereof. Additionally disclosed herein are methods of inhibiting the progression of structural damage in a patient having nr-axSpA, comprising administering an IL-17 antagonist to a patient in need thereof.

In some embodiments of the disclosed uses, methods and kits, the IL-17 antagonist is an IL-17 antibody or antigen-binding fragment thereof. In some embodiments of the disclosed uses, methods and kits, the IL-17 antibody or antigen-binding fragment thereof is selected from the group consisting of: a) an IL-17 antibody or antigen-binding fragment thereof that binds to an epitope of IL-17 comprising Leu74, Tyr85, His86, Met87, Asn88, Val124, Thr125, Pro126, Ile127, Val128, His129; b) an IL-17 antibody or antigen-binding fragment thereof that binds to an epitope of IL-17 comprising Tyr43, Tyr44, Arg46. Ala79, Asp80; c) an IL-17 antibody or antigen-binding fragment thereof that binds to an epitope of an IL-17 homodimer having two mature IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain; d) an IL-17 antibody or antigen-binding fragment thereof that binds to an epitope of an IL-17 homodimer having two mature IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain, wherein the IL-17 antibody or antigen-binding fragment thereof has a K_{D} of about 100-200 pM, and wherein the IL-17 antibody or antigen-binding fragment thereof has an in vivo half-life of about 23 to about 35 days; and e) an IL-17 antibody or antigen-binding fragment thereof comprising: i) an immunoglobulin heavy chain variable domain (V_{H}) comprising the amino acid sequence set forth as SEQ ID NO:8; ii) an immunoglobulin light chain variable domain (V_{L}) comprising the amino acid sequence set forth as SEQ ID NO:10; iii) an immunoglobulin V_{H} domain comprising the amino acid sequence set forth as SEQ ID NO:8 and an immunoglobulin V_{L} domain comprising the amino acid sequence set forth as SEQ ID NO:10; iv) an immunoglobulin V_{H} domain comprising the hypervariable regions set forth as SEQ ID NO: 1, SEQ ID NO:2, and SEQ ID NO:3 v) an immunoglobulin V_{L} domain comprising the hypervariable regions set forth as SEQ ID NO:4, SEQ ID NO:5 and SEQ ID NO:6; vi) an immunoglobulin V_{H} domain comprising the hypervariable regions set forth as SEQ ID NO:11, SEQ ID NO:12 and SEQ ID NO:13; vii) an immunoglobulin V_{H} domain comprising the hypervariable regions set forth as SEQ ID NO: 1, SEQ ID NO:2, and SEQ ID NO:3 and an immunoglobulin V_{L} domain comprising the hypervariable regions set forth as SEQ ID NO:4, SEQ ID NO:5 and SEQ ID NO:6; viii) an immunoglobulin V_{H} domain comprising the hypervariable regions set forth as SEQ ID NO: 11, SEQ ID NO: 12 and SEQ ID NO: 13 and an immunoglobulin V_{L} domain comprising the hypervariable regions set forth as SEQ ID NO:4, SEQ ID NO:5 and SEQ ID NO:6; ix) an immunoglobulin light chain comprising the amino acid sequence set forth as SEQ ID NO: 14; x) an immunoglobulin heavy chain comprising the amino acid sequence set forth as SEQ ID NO: 15; or xi) an immunoglobulin light chain comprising the amino acid sequence set forth as SEQ ID NO: 14 and an immunoglobulin heavy chain comprising the amino acid sequence set forth as SEQ ID NO: 15. In some embodiments of the disclosed uses, methods and kits, the IL-17 antibody or antigen-binding fragment thereof is secukinumab.

### BRIEF DESCRIPTION OF THE FIGURES

FIG. 1. 2009 ASAS classification criteria for axial SpA.

FIG. 2A: At Week 16, improvements from baseline in the mean Berlin SI joint total oedema score were greater for both the secukinumab doses compared with the placebo group. Improvements were sustained through Week 52.

FIG. 2B: Subjects randomized to secukinumab 10 mg/kg IV→150 mg s.c had a lower mean baseline Berlin spine score than the secukinumab 10 mg/kg IV→75 mg s.c and placebo groups. Improvements in the mean Berlin spine score at Week 16 were greater for both secukinumab doses compared with the placebo group. Improvements were sustained through Week 52.

FIG. 3A: Subjects who were switched from placebo to secukinumab at Weeks 16 and 24 showed an improvement in the Berlin SI joint total oedema score at Week 52 from the respective Week 16 scores

FIG. 3B: Subjects who were switched from placebo to secukinumab at Weeks 16 and 24 showed an improvement in the Berlin spine score at Week 52 from the respective Week 16 scores.

### DETAILED DESCRIPTION OF THE DISCLOSURE

As used herein, IL-17 refers to interleukin-17A (IL-17A).

It is an object of the disclosure to provide methods for treating non-radiographic axial spondyloarthritis (nr-axSpA) patients using IL-17 antagonists, e.g., secukinumab. It is another object of the disclosure to provide methods for inhibiting structural damage (e.g., bone and joint) in nr-axSpA patients using IL-17 antagonists, e.g., secukinumab.

The term “comprising” encompasses “including” as well as “consisting,” e.g., a composition “comprising” X may consist exclusively of X or may include something additional, e.g., X+Y.

As used herein, the phrase “inhibiting the progression of structural damage” is synonymous with “preventing the progression of structural damage,” and is used to mean reducing, abrogating or slowing the bone and joint damage that is associated with nr-axSpA. As such, it refers to a decrease in the level and/or rate of progression of damage to the bones and/or joints comprising pathogenic new bone formation of a patient with nr-axSpA. Radiography and Magnetic Resonance Imaging (MRI) are particularly useful tools for analyzing the bone and joint damage associated with axSpA. Various methods of imaging and scoring axial spondyloarthritis may be found in, e.g., Braun and Baraliakos (2011) Ann Rheum Dis 70 (Suppl 1): i97-i103, Rudwaleit (2009) Ann. Rheum. Dis. 68:1520-7; and I-H Song et al. Ann Rheum Dis. 2011 July; 70(7):1257-63. Preferred methods of scoring spine and SIJ MRI images include the Berlin MRI spine score (Lukas C, et al. J Rheumatol. 2007; 34:862-70), the Berlin SU score (Hermann K G, et al. Radiologe. 2004; 44:217-28, Song et al. 2000, supra), the ankylosing spondylitis spine MRI scoring system for disease activity (ASspiMRI-a) and the ‘Berlin modification of ASspiMRI-a’ (Lukas C et al (2007) J. Rheumatol; 34(4):862-70; Rudwaleit et al. (2008) Arthritis Rheum 67:1276-1281; Rudwaleit et al (2005) [abstract] Arthritis Rheum 50:S211). SI joints can also be scored using the Spondyloarthritis Research Consortium of Canada (SPARCC) scoring system (Maksymowych et al. (2005) Arthritis Rheum. 53:703-09). Inhibition can be identified relative to a control, e.g., a patient not treated with the disclosed IL-17 antagonists, or a known rate of progression (e.g., mean, median, or range).

The term “about” in relation to a numerical value x means, for example, +/−10%. When used in front of a numerical range or list of numbers, the term “about” applies to each number in the series, e.g., the phrase “about 1-5” should be interpreted as “about 1-about 5”, or, e.g., the phrase “about 1, 2, 3, 4” should be interpreted as “about 1, about 2, about 3, about 4, etc.”

The word “substantially” does not exclude “completely,” e.g., a composition which is “substantially free” from Y may be completely free from Y. Where necessary, the word “substantially” may be omitted from the definition of the disclosure.

The term “antibody” as referred to herein includes whole antibodies and any antigen-binding portion or single chains thereof. A naturally occurring “antibody” is a glycoprotein comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The V_{H} and V_{L} regions can be further subdivided into regions of hypervariability, termed hypervariable regions or complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding fragment” of an antibody, as used herein, refers to fragments of an antibody that retain the ability to specifically bind to an antigen (e.g., IL-17). It has been shown that the antigen-binding function of an antibody can be performed by fragments of a full-length antibody. Examples of binding fragments encompassed within the term “antigen-binding portion” of an antibody include a Fab fragment, a monovalent fragment consisting of the V_{L}, V_{H}, CL and CH1 domains; a F(ab)2 fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., 1989 Nature 341:544-546), which consists of a V_{H} domain; and an isolated CDR. Exemplary antigen-binding sites include the CDRs of secukinumab as set forth in SEQ ID NOs: 1-6 and 11-13 (Table 1), preferably the heavy chain CDR3. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al., 1988 Science 242:423-426; and Huston et al., 1988 Proc. Natl. Acad. Sci. 85:5879-5883). Such single chain antibodies are also intended to be encompassed within the term “antibody”. Single chain antibodies and antigen-binding portions are obtained using conventional techniques known to those of skill in the art.

An “isolated antibody”, as used herein, refers to an antibody that is substantially free of other antibodies having different antigenic specificities (e.g., an isolated antibody that specifically binds IL-17 is substantially free of antibodies that specifically bind antigens other than IL-17). The term “monoclonal antibody” or “monoclonal antibody composition” as used herein refer to a preparation of antibody molecules of single molecular composition. The term “human antibody”, as used herein, is intended to include antibodies having variable regions in which both the framework and CDR regions are derived from sequences of human origin. A “human antibody” need not be produced by a human, human tissue or human cell. The human antibodies of the disclosure may include amino acid residues not encoded by human sequences (e.g., mutations introduced by random or site-specific mutagenesis in vitro, by N-nucleotide addition at junctions in vivo during recombination of antibody genes, or by somatic mutation in vivo). In some embodiments of the disclosed processes and compositions, the IL-17 antibody is a human antibody, an isolated antibody, and/or a monoclonal antibody.

The term “IL-17” refers to IL-17A, formerly known as CTLA8, and includes wild-type IL-17A from various species (e.g., human, mouse, and monkey), polymorphic variants of IL-17A, and functional equivalents of IL-17A. Functional equivalents of IL-17A according to the present disclosure preferably have at least about 65%, 75%, 85%, 95%, 96%, 97%, 98%, or even 99% overall sequence identity with a wild-type IL-17A (e.g., human IL-17A), and substantially retain the ability to induce IL-6 production by human dermal fibroblasts.

The term “K_{D}” is intended to refer to the dissociation rate of a particular antibody-antigen interaction. The term “K_{D}”, as used herein, is intended to refer to the dissociation constant, which is obtained from the ratio of K_{d} to Kₐ(i.e., K_{d}/Kₐ) and is expressed as a molar concentration (M). K_{D} values for antibodies can be determined using methods well established in the art. A method for determining the K_{D} of an antibody is by using surface plasmon resonance, or using a biosensor system such as a Biacore® system. In some embodiments, the IL-17 antibody or antigen-binding fragment thereof, e.g., secukinumab, binds human IL-17 with a K_{D} of about 100-250 pM.

The term “affinity” refers to the strength of interaction between antibody and antigen at single antigenic sites. Within each antigenic site, the variable region of the antibody “arm” interacts through weak non-covalent forces with antigen at numerous sites; the more interactions, the stronger the affinity. Standard assays to evaluate the binding affinity of the antibodies toward IL-17 of various species are known in the art, including for example, ELISAs, western blots and RIAs. The binding kinetics (e.g., binding affinity) of the antibodies also can be assessed by standard assays known in the art, such as by Biacore analysis.

An antibody that “inhibits” one or more of these IL-17 functional properties (e.g., biochemical, immunochemical, cellular, physiological or other biological activities, or the like) as determined according to methodologies known to the art and described herein, will be understood to relate to a statistically significant decrease in the particular activity relative to that seen in the absence of the antibody (or when a control antibody of irrelevant specificity is present). An antibody that inhibits IL-17 activity affects a statistically significant decrease, e.g., by at least about 10% of the measured parameter, by at least 50%, 80% or 90%, and in certain embodiments of the disclosed methods and compositions, the IL-17 antibody used may inhibit greater than 95%, 98° % or 99% of IL-17 functional activity.

“Inhibit IL-6” as used herein refers to the ability of an IL-17 antibody or antigen-binding fragment thereof (e.g., secukinumab) to decrease IL-6 production from primary human dermal fibroblasts. The production of IL-6 in primary human (dermal) fibroblasts is dependent on IL-17 (Hwang et al., (2004) Arthritis Res Ther; 6:R120-128). In short, human dermal fibroblasts are stimulated with recombinant IL-17 in the presence of various concentrations of an IL-17 binding molecule or human IL-17 receptor with Fc part. The chimeric anti-CD25 antibody Simulect® (basiliximab) may be conveniently used as a negative control. Supernatant is taken after 16 h stimulation and assayed for IL-6 by ELISA. An IL-17 antibody or antigen-binding fragment thereof, e.g., secukinumab, typically has an IC₅₀ for inhibition of IL-6 production (in the presence 1 nM human IL-17) of about 50 nM or less (e.g., from about 0.01 to about 50 nM) when tested as above, i.e., said inhibitory activity being measured on IL-6 production induced by hu-IL-17 in human dermal fibroblasts. In some embodiments of the disclosed methods and compositions, IL-17 antibodies or antigen-binding fragments thereof, e.g., secukinumab, and functional derivatives thereof have an IC₅₀ for inhibition of IL-6 production as defined above of about 20 nM or less, more preferably of about 10 nM or less, more preferably of about 5 nM or less, more preferably of about 2 nM or less, more preferably of about 1 nM or less.

The term “derivative”, unless otherwise indicated, is used to define amino acid sequence variants, and covalent modifications (e.g., pegylation, deamidation, hydroxylation, phosphorylation, methylation, etc.) of an IL-17 antibody or antigen-binding fragment thereof, e.g., secukinumab, according to the present disclosure, e.g., of a specified sequence (e.g., a variable domain). A “functional derivative” includes a molecule having a qualitative biological activity in common with the disclosed IL-17 antibodies. A functional derivative includes fragments and peptide analogs of an IL-17 antibody as disclosed herein. Fragments comprise regions within the sequence of a polypeptide according to the present disclosure, e.g., of a specified sequence. Functional derivatives of the IL-17 antibodies disclosed herein (e.g., functional derivatives of secukinumab) preferably comprise V_{H} and/or V_{L} domains that have at least about 65%, 75%, 85%, 95%, 96%, 97%, 98%, or even 990/% overall sequence identity with the V_{H} and/or V_{L} sequences of the IL-17 antibodies and antigen-binding fragments thereof disclosed herein (e.g., the V_{H} and/or V_{L} sequences of Table 1), and substantially retain the ability to bind human IL-17 or, e.g., inhibit IL-6 production of IL-17 induced human dermal fibroblasts.

The phrase “substantially identical” means that the relevant amino acid or nucleotide sequence (e.g., V_{H} or V_{L} domain) will be identical to or have insubstantial differences (e.g., through conserved amino acid substitutions) in comparison to a particular reference sequence. Insubstantial differences include minor amino acid changes, such as 1 or 2 substitutions in a 5 amino acid sequence of a specified region (e.g., V_{H} or V_{L} domain). In the case of antibodies, the second antibody has the same specificity and has at least 50% of the affinity of the same. Sequences substantially identical (e.g., at least about 85% sequence identity) to the sequences disclosed herein are also part of this application. In some embodiments, the sequence identity of a derivative IL-17 antibody (e.g., a derivative of secukinumab, e.g., a secukinumab biosimilar antibody) can be about 90% or greater, e.g., 90%, 91%, 92%, 93%, 94%, 95%, 96%, 97%, 98%, 99% or higher relative to the disclosed sequences.

“Identity” with respect to a native polypeptide and its functional derivative is defined herein as the percentage of amino acid residues in the candidate sequence that are identical with the residues of a corresponding native polypeptide, after aligning the sequences and introducing gaps, if necessary, to achieve the maximum percent identity, and not considering any conservative substitutions as part of the sequence identity. Neither N- or C-terminal extensions nor insertions shall be construed as reducing identity. Methods and computer programs for the alignment are well known. The percent identity can be determined by standard alignment algorithms, for example, the Basic Local Alignment Search Tool (BLAST) described by Altshul et al. ((1990) J. Mol. Biol., 215: 403 410); the algorithm of Needleman et al. ((1970) J. Mol. Biol., 48: 444 453); or the algorithm of Meyers et al. ((1988) Comput. Appl. Biosci., 4: 11 17). A set of parameters may be the Blosum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4. “Amino acid(s)” refer to all naturally occurring L-α-amino acids, e.g., and include D-amino acids. The phrase “amino acid sequence variant” refers to molecules with some differences in their amino acid sequences as compared to the sequences according to the present disclosure. Amino acid sequence variants of an antibody according to the present disclosure, e.g., of a specified sequence, still have the ability to bind the human IL-17 or, e.g., inhibit IL-6 production of IL-17 induced human dermal fibroblasts. Amino acid sequence variants include substitutional variants (those that have at least one amino acid residue removed and a different amino acid inserted in its place at the same position in a polypeptide according to the present disclosure), insertional variants (those with one or more amino acids inserted immediately adjacent to an amino acid at a particular position in a polypeptide according to the present disclosure) and deletional variants (those with one or more amino acids removed in a polypeptide according to the present disclosure).

The term “pharmaceutically acceptable” means a nontoxic material that does not interfere with the effectiveness of the biological activity of the active ingredient(s).

The term “administering” in relation to a compound, e.g., an IL-17 binding molecule or another agent, is used to refer to delivery of that compound to a patient by any route.

As used herein, a “therapeutically effective amount” refers to an amount of an IL-17 antagonist, e.g., IL-17 binding molecule (e.g., IL-17 antibody or antigen-binding fragment thereof, e.g., secukinumab) or IL-17 receptor binding molecule (e.g., IL-17 antibody or antigen-binding fragment thereof) that is effective, upon single or multiple dose administration to a patient (such as a human) for treating, preventing, preventing the onset of, curing, delaying, reducing the severity of, ameliorating at least one symptom of a disorder or recurring disorder, or prolonging the survival of the patient beyond that expected in the absence of such treatment.

When applied to an individual active ingredient (e.g., an IL-17 antagonist, e.g., secukinumab) administered alone, the term refers to that ingredient alone. When applied to a combination, the term refers to combined amounts of the active ingredients that result in the therapeutic effect, whether administered in combination, serially or simultaneously.

The term “treatment” or “treat” refer to both prophylactic or preventative treatment as well as curative or disease modifying treatment, including treatment of a patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected absent such treatment.

As used herein, the phrases “has not previously been treated with a TNF antagonist” and “TNF Naïve” refer to a nr-axSpA patient who has not been previously treated with a TNF alpha inhibitor for nr-axSpA. As used herein, the phrases “has previously been treated with a TNF antagonist” and “TNF experienced” refer to an nr-axSpA patient who has been previously treated with a TNF alpha inhibitor (e.g., infliximab, etanercept, adalimumab, certolizumab, golimumab). It includes patients who were refractory to or had an inadequate response to TNF alpha inhibitor treatment, as well as patients who stopped treatment with the TNF alpha inhibitor for safety or tolerability reasons. As used herein, the phrases “previously failed to respond to or had an inadequate response to treatment with a TNF alpha antagonist,” “TNF-inadequate responder” and “TNF-IR” refer to an nr-axSpA patient who has been previously treated with a TNF alpha inhibitor for nr-axSpA (e.g., infliximab, etanercept, adalimumab, certolizumab, golimumab), but whose symptoms (e.g., bone and/or joint symptoms) were not adequately controlled by the TNF alpha inhibitor (e.g., a patient with active nr-axSpA despite at least 2 weeks, 4 weeks, at least 8 weeks, at least 3 months, at least 14 weeks, or at least 4 months of treatment using an approved dose of the anti-TNF alpha agent). In some embodiments of the disclosed methods, regimens, uses, kits, and pharmaceutical compositions, the patient previously failed to respond to or had an inadequate response to treatment with a TNF alpha inhibitor.

As used herein, the phrase “previously failed to respond to or had an inadequate response to treatment with nonsteroidal anti-inflammatory drugs (NSAIDs),” refer to an nr-axSpA patient who has been previously treated with on or more NSAIDs for nr-axSpA (e.g., a COX-1 or COX-2 inhibitor), but whose symptoms (e.g., pain, bone and/or joint symptoms) were not adequately controlled by the NSAID (e.g., a patient with active nr-axSpA despite at least 2 weeks, 4 weeks, at least 8 weeks, at least 3 months, at least 14 weeks, or at least 4 months of treatment using an approved dose of the NSAID). In some embodiments of the disclosed methods, regimens, uses, kits, and pharmaceutical compositions, the patient previously failed to respond to or had an inadequate response to treatment with one or more nonsteroidal anti-inflammatory drugs (NSAIDs).

As used herein, “selecting” and “selected” in reference to a patient is used to mean that a particular patient is specifically chosen from a larger group of patients on the basis of (due to) the particular patient having a predetermined criteria. Similarly, “selectively treating” refers to providing treatment to a patient having a particular disease, where that patient is specifically chosen from a larger group of patients on the basis of the particular patient having a predetermined criterion. Similarly, “selectively administering” refers to administering a drug to a patient that is specifically chosen from a larger group of patients on the basis of (due to) the particular patient having a predetermined criterion. By selecting, selectively treating and selectively administering, it is meant that a patient is delivered a personalized therapy based on the patient's personal history (e.g., prior therapeutic interventions, e.g., prior treatment with biologics), biology (e.g., particular genetic markers), and/or manifestation (e.g., not fulfilling particular diagnostic criteria), rather than being delivered a standard treatment regimen based solely on the patient's membership in a larger group. Selecting, in reference to a method of treatment as used herein, does not refer to fortuitous treatment of a patient having a particular criterion, but rather refers to the deliberate choice to administer treatment to a patient based on the patient having a particular criterion. Thus, selective treatment/administration differs from standard treatment/administration, which delivers a particular drug to all patients having a particular disease, regardless of their personal history, manifestations of disease, and/or biology. In some embodiments, the axSpA patient is selected for treatment by fulfilling the ASAS axSpA criteria, while concurrently not satisfying the radiological criterion according to the modified New York diagnostic criteria for ankylosing spondylitis. Patients having this set of characteristics are referred to herein as having “axial spondyloarthritis (axSpA) without radiographic evidence of ankylosing spondylitis” or simply “nr-axSpA”. In some embodiments of the disclosed methods, regimens, uses, kits, and pharmaceutical compositions, the patient has axial spondyloarthritis (axSpA) without radiographic evidence of ankylosing spondylitis (nr-axSpA).

Radiographic changes in the sacroiliac joints of at least grade II or higher bilaterally or grade III or IV unilaterally are a requirement for making a diagnosis of AS according to the modified New York Criteria (Van der Linden et al. (1984) Arthritis Rheum 27:361-8). These changes are referred to herein as “radiological criterion according to the modified New York diagnostic criteria for ankylosing spondylitis” and “radiographic evidence of ankylosing spondylitis.”

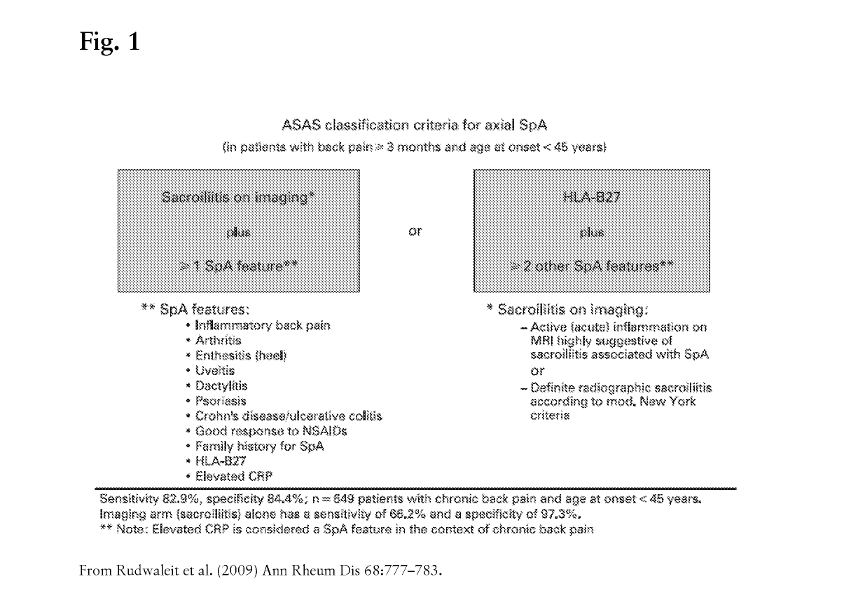

Spondyloarthritides (SpA) is a group of related diseases which comprise ankylosing spondylitis, reactive arthritis, arthritis/spondylitis with inflammatory bowel disease, arthritis/spondylitis with psoriasis, and undifferentiated spondyloarthritis. SpA patients having predominantly axial skeletal symptoms are referred to as having axial SpA (axSpA). The Assessment of SpondyloArthritis international Society (ASAS) criteria has been developed as classification criteria for axial spondyloartiritis (axSpA), covering both radiographic axial SpA and nr-axSpA (Rudwaleit et al. (2009) Ann. Rheum. Dis. 68:777-83, incorporated by reference herein in its entirety). The ASAS axSpA criteria are shown in FIG. 1. In brief, they are: a) the presence of sacroiliitis by radiography (radiographic sacroiliitis according to the modified New York criteria) or by MRI, plus at least one SpA feature (imaging arm); or b) the presence of HLA-B27 plus at least two SpA features (clinical arm). “SpA features” include inflammatory back pain, elevated CRP (in the context of inflammatory back pain), HLA-B27 positive, family history for SpA, good response to NSAIDs, Crohn's disease/ulcerative colitis, psoriasis, dactylitis, uveitis, enthesitis (heel), and arthritis. Patients satisfying the ASAS axSpA criteria, but not having radiographic sacroiliitis according to the modified New York criteria, are referred to as having non-radiographic axial spondyloarthritis (nr-axSpA).

As used herein, a patient is “HLA-B27 positive” if laboratory testing reveals the presence of the HLA-B27 antigen or allele (e.g., using flow cytometry or PCR genotyping).

As used herein, the phrase “inflammatory back pain” refers to back pain that is not mechanical. It is characterized by, e.g., gradual onset, lasting at least 3 months, onset at a relatively young age, alternating buttock pain, morning stiffness lasting for more than 30 minutes, pain at night, lack of improvement with rest, etc. It is not caused by strain or injury and does not tend to develop quickly or have variable onset, and can be diagnosed by a skilled physician.

As used herein, “active nr-axSpA” refers to disease signs and symptoms consistent with a total Bath Ankylosing Spondylitis Disease Activity Index (BASDAI) score of 4 or higher on a scale of 0 to 10. In some embodiments of the disclosed methods, regimens, uses, kits, and pharmaceutical compositions, the patient has active nr-axSpA. In some embodiments of the disclosed methods, regimens, uses, kits, and pharmaceutical compositions, the patient has total BASDAI≥4 cm (0-10 cm) at baseline, spinal pain as measured by BASDAI question #2≥4 cm (0-10 cm) at baseline, and total back pain as measured by VAS≥40 mm (0-100 mm) at baseline.

As used herein, “severe nr-axSpA” and “moderate to severe nr-axSpA” refer to disease signs and symptoms requiring treatment with biologic therapy. According to the “ASAS recommendations for the use of anti-TNF agents in patients with axial spondyloarthritis” (van der Heijde et al (2011) Ann Rheum Dis. 2011 June; 70(6):905-8) patients with nr-axSpA require biologic therapy if they show active disease with a total Bath Ankylosing Spondylitis Disease Activity Index score of 4 or higher on a scale of 0 to 10 after therapy with at least two NSAIDs over a 4-week period in total at the maximum recommended dose unless contraindicated. In some embodiments of the disclosed methods, regimens, uses, kits, and pharmaceutical compositions, the patient has severe nr-axSpA.

As used herein, the phrases “objective signs of inflammation by elevated CRP and/or MRI” and “objective signs of inflammation by CRP and/or MRI” refer to either MRI evidence of sacroiliac joints (SIJ) inflammation, elevated C-reactive protein (CRP), or both. In some embodiments of the disclosed methods, regimens, uses, kits, and pharmaceutical compositions, the patient has axSpA (e.g., severe, moderate-to-severe, active) without radiographic evidence of ankylosing spondylitis, but with objective signs of inflammation as either MRI evidence of sacroiliac joints (SIJ) inflammation and/or elevated C-reactive protein (CRP). Another objective sign of inflammation is inflammation of the spine, which is also observable by MRI. Inflammation of the spine may be scored using the ankylosing spondylitis spine MRI scoring system for disease activity (ASspiMRI-a) and the ‘Berlin modification of ASspiMRI-a’ (Lukas C et al (2007) J. Rheumatol; 34(4):862-70; Rudwaleit et al. (2008) Arthritis Rheum 67:1276-1281; Rudwaleit et al (2005) [abstract] Arthritis Rheum 50:S211).

Recent MRI methodology allows one to demonstrate the presence of active inflammation in the SIJ, the spine and other skeletal elements in patients with axSpA and normal radiographic findings (see, e.g., Rudwaleit et al. (2009) Ann. Rheum Dis. 68:1520-7; Braun et al 1994, Arthritis Rheum 37:1039-45; Oostveen et al 1999, J. Rheumatol. 26:1953-58; Heuft-Dorenbosch et al 2006, Ann. Rheum. Dis. 65:804-08; Heuft-Dorenbosch et al. 2006 Arthritis Res. Ther. 8:R11; Braun and Baraliakos (2011) Ann Rheum Dis 70 (Suppl 1):i97-i103; and for a review, Ambak et al. 2012 Arthritis Res. & Therapy 14:R55), as well as the depiction of acute inflammatory lesions and chronic/structural changes in both the SU and the spine. There are various scoring methods that can be used to identify MRI evidence that is highly suggestive of sacroiliits, which is referred to herein as “MRI evidence of sacroiliac joints (SIJ) inflammation.” A preferred MRI scoring system for use in the disclosed methods is the Berlin SU score (Hermann K G, et al. Radiologe. 2004, 44:217-28). In some embodiments of the disclosed methods, regimens, uses, kits, and pharmaceutical compositions, the patient has MRI evidence of SIJ inflammation.

As used herein “elevated CRP” refers to elevated CRP blood levels, according to an assaying laboratory. An above normal CRP level is defined in the 2010 ACR/EULAR criteria (Aletaha et al. (2010) Ann. Rheum. Dis. 69:1580-88). According to the 2010 ACR/EULAR criteria, normal/abnormal CRP is based on local laboratory standards. Each local laboratory will employ a cutoff value for abnormal (high) CRP based on that laboratory's particular rule for calculating normal maximum CRP. A physician generally orders a CRP test from a local laboratory, and the local laboratory reports normal or abnormal (low or high) CRP using the rule that particular laboratory employs to calculate normal CRP. In some cases, the laboratory simply reports that the CRP is beyond the “upper limit of normal (ULN).” Thus, unless the context dictates otherwise, as used herein “elevated CRP” is not meant to denote a particular numerical value, as what is considered a normal CRP value will differ between laboratories and assays. In some embodiments of the disclosure, CRP is measured using a high sensitivity assay; elevated CRP by this assay (i.e., hsCRP) can be, e.g., >about 3 mg/L (e.g., 3 mg/L), >about 10 mg/L (e.g., 10 mg/L), >about 20 mg/L (e.g., 20 mg/L) or >about 30 mg/L (e.g., 30 mg/L). The CRP level, when assessed at baseline, is referred to as “baseline CRP”. An elevated level of CRP at baseline may be referred to as “elevated baseline CRP”. In some embodiments of the disclosed methods, regimens, uses, kits, and pharmaceutical compositions, the patient has a high baseline CRP or hsCRP.

### IL-17 Antagonists

The various disclosed processes, kits, uses and methods utilize an IL-17 antagonist, e.g., IL-17 binding molecule (e.g., soluble IL-17 receptor, IL-17 antibody or antigen-binding fragment thereof, e.g., secukinumab) or IL-17 receptor binding molecule (e.g., IL-17 receptor antibody or antigen-binding fragment thereof). In some embodiments, the IL-17 antagonist is an IL-17 binding molecule, preferably an IL-17 antibody or antigen-binding fragment thereof.

In one embodiment, the IL-17 antibody or antigen-binding fragment thereof comprises at least one immunoglobulin heavy chain variable domain (V_{H}) comprising hypervariable regions CDR1, CDR2 and CDR3, said CDR1 having the amino acid sequence SEQ ID NO: 1, said CDR2 having the amino acid sequence SEQ ID NO:2, and said CDR3 having the amino acid sequence SEQ ID NO:3. In one embodiment, the IL-17 antibody or antigen-binding fragment thereof comprises at least one immunoglobulin light chain variable domain (V_{L}) comprising hypervariable regions CDR1′, CDR2′ and CDR3′, said CDR1′ having the amino acid sequence SEQ ID NO:4, said CDR2′ having the amino acid sequence SEQ ID NO:5 and said CDR3′ having the amino acid sequence SEQ ID NO:6. In one embodiment, the IL-17 antibody or antigen-binding fragment thereof comprises at least one immunoglobulin heavy chain variable domain (V_{H}) comprising hypervariable regions CDR1-x, CDR2-x and CDR3-x, said CDR1-x having the amino acid sequence SEQ ID NO: 11, said CDR2-x having the amino acid sequence SEQ ID NO: 12, and said CDR3-x having the amino acid sequence SEQ ID NO:13.

In one embodiment, the IL-17 antibody or antigen-binding fragment thereof comprises at least one immunoglobulin V_{H} domain and at least one immunoglobulin V_{L} domain, wherein: a) the immunoglobulin V_{H} domain comprises (e.g., in sequence): i) hypervariable regions CDR1, CDR2 and CDR3, said CDR1 having the amino acid sequence SEQ ID NO: 1, said CDR2 having the amino acid sequence SEQ ID NO:2, and said CDR3 having the amino acid sequence SEQ ID NO:3, or ii) hypervariable regions CDR1-x, CDR2-x and CDR3-x, said CDR1-x having the amino acid sequence SEQ ID NO: 11, said CDR2-x having the amino acid sequence SEQ ID NO: 12, and said CDR3-x having the amino acid sequence SEQ ID NO:13; and b) the immunoglobulin V_{L} domain comprises (e.g., in sequence) hypervariable regions CDR1′, CDR2′ and CDR3′, said CDR1′ having the amino acid sequence SEQ ID NO:4, said CDR2′ having the amino acid sequence SEQ ID NO:5, and said CDR3′ having the amino acid sequence SEQ ID NO:6.

In one embodiment, the IL-17 antibody or antigen-binding fragment thereof comprises: a) an immunoglobulin heavy chain variable domain (V_{H}) comprising the amino acid sequence set forth as SEQ ID NO:8; b) an immunoglobulin light chain variable domain (V_{L}) comprising the amino acid sequence set forth as SEQ ID NO:10; c) an immunoglobulin V_{H} domain comprising the amino acid sequence set forth as SEQ ID NO:8 and an immunoglobulin V_{L} domain comprising the amino acid sequence set forth as SEQ ID NO: 10; d) an immunoglobulin V_{H} domain comprising the hypervariable regions set forth as SEQ ID NO:1, SEQ ID NO:2, and SEQ ID NO:3; e) an immunoglobulin V_{L} domain comprising the hypervariable regions set forth as SEQ ID NO:4. SEQ ID NO:5 and SEQ ID NO:6; f) an immunoglobulin V_{H} domain comprising the hypervariable regions set forth as SEQ ID NO:11, SEQ ID NO:12 and SEQ ID NO:13; g) an immunoglobulin V_{H} domain comprising the hypervariable regions set forth as SEQ ID NO: 1, SEQ ID NO:2, and SEQ ID NO:3 and an immunoglobulin V_{L} domain comprising the hypervariable regions set forth as SEQ ID NO:4, SEQ ID NO:5 and SEQ ID NO:6. or h) an immunoglobulin V_{H} domain comprising the hypervariable regions set forth as SEQ ID NO: 11, SEQ ID NO: 12 and SEQ ID NO: 13 and an immunoglobulin V_{L} domain comprising the hypervariable regions set forth as SEQ ID NO:4, SEQ ID NO:5 and SEQ ID NO:6.

For ease of reference the amino acid sequences of the hypervariable regions of the secukinumab monoclonal antibody, based on the Kabat definition and as determined by the X-ray analysis and using the approach of Chothia and coworkers, is provided in Table 1, below.

TABLE 1
Amino acid sequences of the hypervariable regions of secukinumabo
Light-Chain

| | | |
|---|---|---|
| CDR1′ | Kabat | R-A-S-Q-S-V-S-S-S-Y-L-A (SEQ ID NO: 4) |
| | Chothia | R-A-S-Q-S-V-S-S-S-Y-L-A (SEQ ID NO: 4) |
| CDR2′ | Kabat | G-A-S-S-R-A-T (SEQ ID NO: 5) |
| | Chothia | G-A-S-S-R-A-T (SEQ ID NO: 5) |
| CDR2′ | Kabat | Q-Q-Y-G-S-S-P-C-T (SEQ ID NO: 6) |
| | Chothia | Q-Q-Y-G-S-S-P-C-T (SEQ ID NO: 6) |

Heavy-Chain

| | | |
|---|---|---|
| CDR1 | Kabat | N-Y-W-M-N (SEQ ID NO: 1) |
| CDR1-x | Chothia | G-F-T-F-S-N-Y-W-M-N (SEQ ID NO: 11) |
| CDR2 | Kabat | A-I-N-Q-D-G-S-E-K-Y-Y-V-G-S-V-K-G (SEQ ID NO: 2) |
| CDR2-x | Chothia | A-I-N-Q-D-G-S-E-K-Y-Y (SEQ ID NO: 12) |
| CDR3 | Kabat | D-Y-Y-D-I-L-T-D-Y-Y-I-H-Y-W-Y-F-D-L (SEQ ID |
| | | NO: 3) |
| CDR3-x | Chothia | C-V-R-D-Y-Y-D-I-L-T-D-Y-Y-I-H-Y-W-Y-F-D-L-W-G |
| | | (SEQ ID NO: 13) |

In preferred embodiments, the constant region domains preferably also comprise suitable human constant region domains, for instance as described in “Sequences of Proteins of Immunological Interest”, Kabat E. A. et al, US Department of Health and Human Services, Public Health Service, National Institute of Health. The DNA encoding the VL of secukinumab is set forth in SEQ ID NO:9. The DNA encoding the V_{H} of secukinumab is set forth in SEQ ID NO:7.

In some embodiments, the IL-17 antibody or antigen-binding fragment thereof (e.g., secukinumab) comprises the three CDRs of SEQ ID NO:10. In other embodiments, the IL-17 antibody or antigen-binding fragment thereof comprises the three CDRs of SEQ ID NO:8. In other embodiments, the IL-17 antibody or antigen-binding fragment thereof comprises the three CDRs of SEQ ID NO:10 and the three CDRs of SEQ ID NO:8. CDRs of SEQ ID NO:8 and SEQ ID NO:10 may be found in Table 1. The free cysteine in the light chain (CysL97) may be seen in SEQ ID NO:6.

In some embodiments, IL-17 antibody or antigen-binding fragment thereof comprises the light chain of SEQ ID NO:14. In other embodiments, the IL-17 antibody or antigen-binding fragment thereof comprises the heavy chain of SEQ ID NO:15. In other embodiments, the IL-17 antibody or antigen-binding fragment thereof comprises the light chain of SEQ ID NO:14 and the heavy domain of SEQ ID NO:15. In some embodiments, the IL-17 antibody or antigen-binding fragment thereof comprises the three CDRs of SEQ ID NO:14. In other embodiments, IL-17 antibody or antigen-binding fragment thereof comprises the three CDRs of SEQ ID NO:15. In other embodiments, the IL-17 antibody or antigen-binding fragment thereof comprises the three CDRs of SEQ ID NO:14 and the three CDRs of SEQ ID NO:15. CDRs of SEQ ID NO:14 and SEQ ID NO:15 may be found in Table 1.

Hypervariable regions may be associated with any kind of framework regions, though preferably are of human origin. Suitable framework regions are described in Kabat E. A. et al, ibid. The preferred heavy chain framework is a human heavy chain framework, for instance that of the secukinumab antibody. It consists in sequence, e.g. of FR1 (amino acid 1 to 30 of SEQ ID NO:8), FR2 (amino acid 36 to 49 of SEQ ID NO:8), FR3 (amino acid 67 to 98 of SEQ ID NO:8) and FR4 (amino acid 117 to 127 of SEQ ID NO:8) regions. Taking into consideration the determined hypervariable regions of secukinumab by X-ray analysis, another preferred heavy chain framework consists in sequence of FR1-x (amino acid 1 to 25 of SEQ ID NO:8), FR2-x (amino acid 36 to 49 of SEQ ID NO:8), FR3-x (amino acid 61 to 95 of SEQ ID NO:8) and FR4 (amino acid 119 to 127 of SEQ ID NO:8) regions. In a similar manner, the light chain framework consists, in sequence, of FR1′ (amino acid 1 to 23 of SEQ ID NO:10), FR2′ (amino acid 36 to 50 of SEQ ID NO:10), FR3′ (amino acid 58 to 89 of SEQ ID NO: 10) and FR4′ (amino acid 99 to 109 of SEQ ID NO:10) regions.

In one embodiment, the IL-17 antibody or antigen-binding fragment thereof (e.g., secukinumab) is selected from a human IL-17 antibody that comprises at least: a) an immunoglobulin heavy chain or fragment thereof which comprises a variable domain comprising, in sequence, the hypervariable regions CDR1, CDR2 and CDR3 and the constant part or fragment thereof of a human heavy chain; said CDR1 having the amino acid sequence SEQ ID NO:1, said CDR2 having the amino acid sequence SEQ ID NO:2, and said CDR3 having the amino acid sequence SEQ ID NO:3; and b) an immunoglobulin light chain or fragment thereof which comprises a variable domain comprising, in sequence, the hypervariable regions CDR1′, CDR2′, and CDR3′ and the constant part or fragment thereof of a human light chain, said CDR1′ having the amino acid sequence SEQ ID NO:4, said CDR2′ having the amino acid sequence SEQ ID NO:5, and said CDR3′ having the amino acid sequence SEQ ID NO:6.

In one embodiment, the IL-17 antibody or antigen-binding fragment thereof is selected from a single chain antibody or antigen-binding fragment thereof that comprises an antigen-binding site comprising: a) a first domain comprising, in sequence, the hypervariable regions CDR1, CDR2 and CDR3, said CDR1 having the amino acid sequence SEQ ID NO:1, said CDR2 having the amino acid sequence SEQ ID NO:2, and said CDR3 having the amino acid sequence SEQ ID NO:3; and b) a second domain comprising, in sequence, the hypervariable regions CDR1′, CDR2′ and CDR3′, said CDR1′ having the amino acid sequence SEQ ID NO:4, said CDR2′ having the amino acid sequence SEQ ID NO:5, and said CDR3′ having the amino acid sequence SEQ ID NO:6, and c) a peptide linker which is bound either to the N-terminal extremity of the first domain and to the C-terminal extremity of the second domain or to the C-terminal extremity of the first domain and to the N-terminal extremity of the second domain.

Alternatively, an IL-17 antibody or antigen-binding fragment thereof as used in the disclosed methods may comprise a derivative of the IL-17 antibodies set forth herein by sequence (e.g., a pegylated version of secukinumab). Alternatively, the V_{H} or V_{L} domain of an IL-17 antibody or antigen-binding fragment thereof used in the disclosed methods may have V_{H} or V_{L} domains that are substantially identical to the V_{H} or V_{L} domains set forth herein (e.g., those set forth in SEQ ID NO:8 and 10). A human IL-17 antibody disclosed herein may comprise a heavy chain that is substantially identical to that set forth as SEQ ID NO: 15 and/or a light chain that is substantially identical to that set forth as SEQ ID NO: 14. A human IL-17 antibody disclosed herein may comprise a heavy chain that comprises SEQ ID NO: 15 and a light chain that comprises SEQ ID NO:14. A human IL-17 antibody disclosed herein may comprise: a) one heavy chain which comprises a variable domain having an amino acid sequence substantially identical to that shown in SEQ ID NO:8 and the constant part of a human heavy chain; and b) one light chain which comprises a variable domain having an amino acid sequence substantially identical to that shown in SEQ ID NO:10 and the constant part of a human light chain.

Alternatively, an IL-17 antibody or antigen-binding fragment thereof used in the disclosed methods may be an amino acid sequence variant of the reference IL-17 antibodies set forth herein, as long as it contains CysL97. The disclosure also includes IL-17 antibodies or antigen-binding fragments thereof (e.g., secukinumab) in which one or more of the amino acid residues of the V_{H} or V_{L} domain of secukinumab (but not CysL97), typically only a few (e.g., 1-10), are changed; for instance by mutation, e.g., site directed mutagenesis of the corresponding DNA sequences. In all such cases of derivative and variants, the IL-17 antibody or antigen-binding fragment thereof is capable of inhibiting the activity of about 1 nM (=30 ng/ml) human IL-17 at a concentration of about 50 nM or less, about 20 nM or less, about 10 nM or less, about 5 nM or less, about 2 nM or less, or more preferably of about 1 nM or less of said molecule by 50%, said inhibitory activity being measured on IL-6 production induced by hu-IL-17 in human dermal fibroblasts as described in Example 1 of WO 2006/013107.

In some embodiments, the IL-17 antibodies or antigen-binding fragments thereof, e.g., secukinumab, bind to an epitope of mature human IL-17 comprising Leu74, Tyr85, His86, Met87, Asn88, Val124, Thr125, Pro126, Ile127, Val128, His129. In some embodiments, the IL-17 antibody, e.g., secukinumab, binds to an epitope of mature human IL-17 comprising Tyr43, Tyr44, Arg46, Ala79, Asp80. In some embodiments, the IL-17 antibody, e.g., secukinumab, binds to an epitope of an IL-17 homodimer having two mature human IL-17 chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain. The residue numbering scheme used to define these epitopes is based on residue one being the first amino acid of the mature protein (i.e., IL-17A lacking the 23 amino acid N-terminal signal peptide and beginning with Glycine). The sequence for immature IL-17A is set forth in the Swiss-Prot entry Q16552. In some embodiments, the IL-17 antibody has a K_{D} of about 100-200 pM. In some embodiments, the IL-17 antibody has an IC₅₀ of about 0.4 nM for in vitro neutralization of the biological activity of about 0.67 nM human IL-17A. In some embodiments, the absolute bioavailability of subcutaneously (SC) administered IL-17 antibody has a range of about 60-about 80%, e.g., about 76%. In some embodiments, the IL-17 antibody, such as secukinumab, has an elimination half-life of about 4 weeks (e.g., about 23 to about 35 days, about 23 to about 30 days, e.g., about 30 days). In some embodiments, the IL-17 antibody (such as secukinumab) has a Tm, of about 7-8 days.

Particularly preferred IL-17 antibodies or antigen-binding fragments thereof used in the disclosed methods are human antibodies, especially secukinumab as described in Examples 1 and 2 of WO 2006/013107. Secukinumab is a recombinant high-affinity, fully human monoclonal anti-human interleukin-17A (IL-17A, IL-17) antibody of the IgG1/kappa isotype that is currently in clinical trials for the treatment of immune-mediated inflammatory conditions. Secukinumab (see, e.g., WO2006/013107 and WO2007/117749) has a very high affinity for IL-17, i.e., a K_{D} of about 100-200 pM and an IC₅₀ for in vitro neutralization of the biological activity of about 0.67 nM human IL-17A of about 0.4 nM. Thus, secukinumab inhibits antigen at a molar ratio of about 1:1. This high binding affinity makes the secukinumab antibody particularly suitable for therapeutic applications. Furthermore, it has been determined that secukinumab has a very long half-life, i.e., about 4 weeks, which allows for prolonged periods between administration, an exceptional property when treating chronic life-long disorders, such as nr-axSpA.

Other preferred IL-17 antibodies for use in the disclosed methods, kits and regimens are those set forth in U.S. Pat. Nos. 8,057,794; 8,003,099; 8,110,191; and 7,838,638 and US Published Patent Application Nos: 20120034656 and 20110027290, which are incorporated by reference herein in their entirety.

### Methods of Treatment and Uses of IL-17 Antagonists for Nr-axSpA

The disclosed IL-17 antagonists, e.g., IL-17 binding molecules (e.g., IL-17 antibody or antigen-binding fragment thereof, e.g., secukinumab) or IL-17 receptor binding molecules (e.g., IL-17 receptor antibody or antigen-binding fragment thereof), may be used in vitro, ex vivo, or incorporated into pharmaceutical compositions and administered in vivo to treat nr-axSpA patients (e.g., human patients) and/or to inhibit the progression of structural damage in nr-axSpA patients, e.g., nr-axSpA patients that have not previously been treated with a TNF alpha inhibitor (TNF-naïve patients), nr-axSpA patients that have been previously treated with a TNF alpha inhibitor, e.g., nr-axSpA patients having been treated with a TNF alpha inhibitor, but who had an inadequate response (e.g., failed or less than desirable) thereto (TNF-IR patients), and nr-axSpA patients that have been previously treated with an NSAID but who had an inadequate response (e.g., failed or less than desirable) thereto.

The IL-17 antagonists, e.g., IL-17 binding molecules (e.g., IL-17 antibody or antigen-binding fragment thereof, e.g., secukinumab) or IL-17 receptor binding molecules (e.g., IL-17 antibody or antigen-binding fragment thereof), may be used as a pharmaceutical composition when combined with a pharmaceutically acceptable carrier. Such a composition may contain, in addition to an IL-17 antagonist, carriers, various diluents, fillers, salts, buffers, stabilizers, solubilizers, and other materials well known in the art. The characteristics of the carrier will depend on the route of administration. The pharmaceutical compositions for use in the disclosed methods may also contain additional therapeutic agents for treatment of the particular targeted disorder. For example, a pharmaceutical composition may also include anti-inflammatory agents. Such additional factors and/or agents may be included in the pharmaceutical composition to produce a synergistic effect with the IL-17 binding molecules, or to minimize side effects caused by the IL-17 antagonists, e.g., IL-17 binding molecules (e.g., IL-17 antibody or antigen-binding fragment thereof, e.g., secukinumab) or IL-17 receptor binding molecules (e.g., IL-17 antibody or antigen-binding fragment thereof).

Pharmaceutical compositions for use in the disclosed methods may be manufactured in conventional manner. In one embodiment, the pharmaceutical composition is provided in lyophilized form. For immediate administration it is dissolved in a suitable aqueous carrier, for example sterile water for injection or sterile buffered physiological saline. If it is considered desirable to make up a solution of larger volume for administration by infusion rather than a bolus injection, may be advantageous to incorporate human serum albumin or the patient's own heparinised blood into the saline at the time of formulation. The presence of an excess of such physiologically inert protein prevents loss of antibody by adsorption onto the walls of the container and tubing used with the infusion solution. If albumin is used, a suitable concentration is from 0.5 to 4.5% by weight of the saline solution. Other formulations comprise liquid or lyophilized formulation.

Antibodies, e.g., antibodies to IL-17, are typically formulated either in aqueous form ready for parenteral administration or as lyophilisates for reconstitution with a suitable diluent prior to administration. In some embodiments of the disclosed methods and uses, the IL-17 antagonist, e.g., IL-17 antibody, e.g., secukinumab, is formulated as a lyophilisate. Suitable lyophilisate formulations can be reconstituted in a small liquid volume (e.g., 2 ml or less) to allow subcutaneous administration and can provide solutions with low levels of antibody aggregation. The use of antibodies as the active ingredient of pharmaceuticals is now widespread, including the products HERCEPTIN™ (trastuzumab), RITUXAN™ (rituximab), SYNAGIS™ (palivizumab), etc. Techniques for purification of antibodies to a pharmaceutical grade are well known in the art. When a therapeutically effective amount of an IL-17 antagonist, e.g., IL-17 binding molecules (e.g., IL-17 antibody or antigen-binding fragment thereof, e.g., secukinumab) or IL-17 receptor binding molecules (e.g., IL-17 antibody or antigen-binding fragment thereof) is administered by intravenous, cutaneous or subcutaneous injection, the IL-17 antagonist will be in the form of a pyrogen-free, parenterally acceptable solution. A pharmaceutical composition for intravenous, cutaneous, or subcutaneous injection may contain, in addition to the IL-17 antagonist, an isotonic vehicle such as sodium chloride, Ringer's solution, dextrose, dextrose and sodium chloride, lactated Ringer's solution, or other vehicle as known in the art.

The appropriate dosage will, of course, vary depending upon, for example, the particular IL-17 antagonists, e.g., IL-17 binding molecules (e.g., IL-17 antibody or antigen-binding fragment thereof, e.g., secukinumab) or IL-17 receptor binding molecules (e.g., IL-17 antibody or antigen-binding fragment thereof) to be employed, the host, the mode of administration and the nature and severity of the condition being treated, and on the nature of prior treatments that the patient has undergone. Ultimately, the attending health care provider will decide the amount of the IL-17 antagonist with which to treat each individual patient. In some embodiments, the attending health care provider may administer low doses of the IL-17 antagonist and observe the patient's response. In other embodiments, the initial dose(s) of IL-17 antagonist administered to a patient are high, and then are titrated downward until signs of relapse occur. Larger doses of the IL-17 antagonist may be administered until the optimal therapeutic effect is obtained for the patient, and the dosage is not generally increased further.

In practicing some of the methods of treatment or uses of the present disclosure, a therapeutically effective amount of an IL-17 antagonist, e.g., IL-17 binding molecule (e.g., IL-17 antibody or antigen-binding fragment thereof, e.g., secukinumab) or IL-17 receptor binding molecule (e.g., IL-17 antibody or antigen-binding fragment thereof) is administered to a patient, e.g., a mammal (e.g., a human). While it is understood that the disclosed methods provide for treatment of nr-axSpA patients using an IL-17 antagonist (e.g., secukinumab), this does not preclude that, if the patient is to be ultimately treated with an IL-17 antagonist, such IL-17 antagonist therapy is necessarily a monotherapy. Indeed, if a patient is selected for treatment with an IL-17 antagonist, then the IL-17 antagonist (e.g., secukinumab) may be administered in accordance with the methods of the disclosure either alone or in combination with other agents and therapies for treating nr-axSpA patients, e.g., in combination with at least one additional nr-axSpA agent, such as an immunosuppressive agent, a disease-modifying anti-rheumatic drug (DMARD) (e.g., sulfasalazine), a pain-control drug, a steroid, a non-steroidal anti-inflammatory drug (NSAID), a cytokine antagonist, a bone anabolic, a bone anti-resorptive, and combinations thereof (e.g., dual and triple therapies). When coadministered with one or more additional nr-axSpA agents, an IL-17 antagonist may be administered either simultaneously with the other agent, or sequentially. If administered sequentially, the attending physician will decide on the appropriate sequence of administering the IL-17 antagonist in combination with other agents and the appropriate dosages for co-delivery.

Non-steroidal anti-inflammatory drugs (NSAIDs) and pain control agents useful in combination with secukinumab for the treatment of nr-axSpA patients include, but are not limited to, propionic acid derivative, acetic acid derivative, enolic acid derivatives, fenamic acid derivatives, Cox inhibitors, e.g., lumiracoxib, ibuprofen, fenoprofen, ketoprofen, flurbiprofen, oxaprozin, indomethacin, sulindac, etodolac, ketorolac, nabumetone, aspirin, naproxen, valdecoxib, etoricoxib, MK0966, rofecoxib, acetaminophen, celecoxib, diclofenac, tramadol, piroxicam, meloxicam, tenoxicam, droxicam, lornoxicam, isoxicam, mefanamic acid, meclofenamic acid, flufenamic acid, tolfenamic, parecoxib, firocoxib. DMARDs useful in combination with an IL-17 antagonist, e.g., secukinumab, for the treatment of nr-axSpA patients include, but are not limited to, methotrexate (MTX), antimalarial drugs (e.g., hydroxychloroquine and chloroquine), sulfasalazine, leflunomide, azathioprine, cyclosporin, gold salts, minocycline, cyclophosphamide, D-penicillamine, minocycline, auranofin, tacrolimus, myocrisin, chlorambucil. Steroids (e.g., glucocorticoids) useful in combination with an IL-17 antagonist, e.g., secukinumab, for the treatment of a nr-axSpA patient include, but are not limited to, prednisolone, prednisone, dexamethasone, cortisol, cortisone, hydrocortisone, methylprednisolone, betamethasone, triamcinolone, beclometasone, fludrocortisone, deoxycorticosterone, aldosterone.

Biologic agents that may be useful in combination with an IL-17 antagonist, e.g., secukinumab, for the treatment of an nr-axSpA patient include, but are not limited to, ADALIMUMAB (Humira®), ETANERCEPT (Enbrel®), INFLIXIMAB (Remicade®; TA-650), CERTOLIZUMAB PEGOL (Cimzia®; CDP870), GOLIMUMAB (Simponi®; CNTO148), RITUXIMAB (Rituxan®; MabThera®), ABATACEPT (Orencia®), TOCILIZUMAB (RoActemAS/Actemra®), integrin antagonists (TYSABRI® (natalizumab)), IL-1 antagonists (ACZ885, Canakinumab (Ilaris®), anakinra (Kineret®)), CD4 antagonists, other IL-17 antagonists (LY2439821, ixekizumab, RG4934, AMG827, brodalumab, SCH900117, R05310074, MEDI-571, CAT-2200, IL-23 antagonists, IL-20 antagonists, IL-6 antagonists, other TNF alpha antagonists (e.g., other TNF alpha antagonists or TNF alpha receptor antagonsits, e.g., pegsunercept, etc.), BLyS antagonists (e.g., Atacicept, Benlysta®/LymphoStat-B® (belimumab)), P38 Inhibitors, CD20 antagonists (Ocrelizumab, Ofatumumab (Arzerra®)), Interferon gamma antagonists (Fontolizumab) or biosimilar versions of these biologic agents.

An IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) is conveniently administered parenterally, e.g., intravenously (e.g., into the antecubital or other peripheral vein), intramuscularly, or subcutaneously. The duration of intravenous (IV) therapy using a pharmaceutical composition of the present disclosure will vary, depending on the severity of the disease being treated and the condition and personal response of each individual patient. Also contemplated is subcutaneous (SC) therapy using a pharmaceutical composition of the present disclosure. The health care provider will decide on the appropriate duration of IV or SC therapy and the timing of administration of the therapy, using the pharmaceutical composition of the present disclosure.

Preferred dosing and treatment regimens (including both induction and maintenance regimens) for treating nr-axSpA patients are provided in PCT Application No. PCT/US2011/064307, which is incorporated by reference herein in its entirety.

The IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) may be administered to the nr-axSpA patient intravenously (IV) at about 10 mg/kg every other week during week 0, 2, and 4 and thereafter administered to the patient subcutaneously (SC) at about 75 mg-about 300 mg (e.g., about 75 mg, about 150 mg, about 300 mg) monthly, beginning during week 8. In this manner, the patient is dosed IV with about 10 mg/kg during week 0, 2, 4, and then the patient is dosed SC with about 75 mg-about 300 mg (e.g., about 75 mg, about 150 mg, about 300 mg) of the IL-17 antagonist (e.g., secukinumab) during week 8, 12, 16, 20, etc.

The IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) may be administered to the patient SC at about 75 mg-about 300 mg (e.g., about 75 mg, about 150 mg, about 300 mg) weekly during weeks 0, 1, 2, and 3. and thereafter administered to the patient SC at about 75 mg-about 300 mg (e.g., about 75 mg, about 150 mg, about 300 mg) monthly, beginning during week 4. In this manner, the patient is dosed SC with about 75 mg-about 300 mg (e.g., about 75 mg, about 150 mg, about 300 mg) of the IL-17 antagonist (e.g., secukinumab) during weeks 0, 1, 2, 3, 4, 8, 12, 16, 20, etc.

Alternatively, the IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) may be administered to the patient without a loading regimen, e.g., the antagonist may be administered to the patient SC at about 75 mg-about 300 mg (e.g., about 75 mg, about 150 mg, about 300 mg) every 4 weeks (monthly). In this manner, the patient is dosed SC with about 75 mg-about 300 mg (e.g., about 75 mg, about 150 mg, about 300 mg) of the IL-17 antagonist (e.g., secukinumab) during weeks 0, 4, 8, 12, 16, 20, etc.

It will be understood that dose escalation may be required (e.g., during an induction and/or maintenance phase) for certain patients, e.g., patients that display inadequate response to treatment with the IL-17 antagonists, e.g., IL-17 binding molecules (e.g., IL-17 antibody or antigen-binding fragment thereof, e.g., secukinumab) or IL-17 receptor binding molecules (e.g., IL-17 receptor antibody or antigen-binding fragment thereof). Thus, SC dosages of secukinumab may be greater than about 75 mg to about 300 mg SC, e.g., about 80 mg, about 100 mg. about 125 mg, about 175 mg, about 200 mg, about 250 mg, about 350 mg, about 400 mg, etc.; similarly, IV dosages may be greater than about 10 mg/kg, e.g., about 11 mg/kg, 12 mg/kg, 15 mg/kg, 20 mg/kg, 25 mg/kg, 30 mg/kg, 35 mg/kg, etc. It will also be understood that dose reduction may also be required (e.g., during the induction and/or maintenance phase) for certain patients, e.g., patients that display adverse events or an adverse response to treatment with the IL-17 antagonist (e.g., IL-17 antibody or antigen-binding fragment thereof, e.g., secukinumab). Thus, dosages of the IL-17 antagonist (e.g., IL-17 antibody or antigen-binding fragment thereof, e.g., secukinumab), may be less than about 75 mg to about 300 mg SC, e.g., about 25 mg, about 50 mg, about 80 mg. about 100 mg, about 125 mg, about 175 mg, about 200 mg, 250 mg, etc.; similarly, IV dosages may be less than about 10 mg/kg, e.g., about 9 mg/kg, 8 mg/kg, 5 mg/kg, 4 mg/kg, 3 mg/kg, 2 mg/kg, 1 mg/kg, etc. In some embodiments, the IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) may be administered to the patient at an initial dose of 75 mg delivered SC, and the dose is then escalated to 150 mg or 300 mg if needed, as determined by a physician.

The timing of dosing is generally measured from the day of the first dose of secukinumab (which is also known as “baseline”). However, health care providers often use different naming conventions to identify dosing schedules, as shown in Table 2.

TABLE 2
Common naming conventions for dosing regimens. Bolded
items refer to the naming convention used herein.

| Week | 0/1 | 1/2 | 2/3 | 3/4 | 4/5 | 5/6 | 6/7 | 7/8 | 8/9 | 9/10 | 10/11 | etc |
|---|---|---|---|---|---|---|---|---|---|---|---|---|
| 1^{st} day | 0/1 | 7/8 | 14/15 | 21/22 | 28/29 | 35/36 | 42/43 | 49/50 | 56/57 | 63/64 | 70/71 | etc. |
| of week | | | | | | | | | | | | |

Notably, week zero may be referred to as week one by some health care providers, while day zero may be referred to as day one by some health care providers. Thus, it is possible that different physicians will designate, e.g., a dose as being given during week 3/on day 21, during week 3/on day 22, during week 4/on day 21, during week 4/on day 22, while referring to the same dosing schedule. For consistency, the first week of dosing will be referred to herein as week 0, while the first day of dosing will be referred to as day 1. However, it will be understood by a skilled artisan that this naming convention is simply used for consistency and should not be construed as limiting, i.e., weekly dosing is the provision of a weekly dose of the IL-17 antibody regardless of whether the physician refers to a particular week as “week 1′” or “week 2”. Moreover, in a preferred dosing regimen, the antibody is administered during week 0, 1, 2, 3, 4 8, 12, 16, 20, etc. Some providers may refer to this regimen as weekly for five weeks and then monthly (or every 4 weeks) thereafter, beginning during week 8, while others may refer to this regimen as weekly for four weeks and then monthly (or every 4 weeks) thereafter, beginning during week 4. Thus, it will be appreciated by a skilled artisan that administering a patient an injection at weeks 0, 1, 2 and 3, followed by once monthly dosing starting at week 4 is the same as administering the patient an injection at weeks 0, 1, 2, 3, and 4, followed by once monthly dosing starting at week 8.

Disclosed herein are methods of treating a patient having non-radiographic axial spondyloarthritis (nr-axSpA), comprising administering an IL-17 antibody or antigen-binding fragment thereof to a patient in need thereof, wherein the IL-17 antibody or antigen-binding fragment thereof binds to an epitope of an IL-17 homodimer having two mature IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain, wherein the IL-17 antibody or antigen-binding fragment thereof has a K_{D} of about 100-200 pM, and wherein the IL-17 antibody or antigen-binding fragment thereof has an in vivo half-life of about 4 weeks.

Additionally disclosed herein are methods of inhibiting the progression of structural damage in a patient having nr-axSpA, comprising administering an IL-17 antibody or antigen-binding fragment thereof to a patient in need thereof, wherein the IL-17 antibody or antigen-binding fragment thereof binds to an epitope of an IL-17 homodimer having two mature IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain, wherein the IL-17 antibody or antigen-binding fragment thereof has a K_{D} of about 100-200 pM, and wherein the IL-17 antibody or antigen-binding fragment thereof has an in vivo half-life of about 4 weeks.

Additionally disclosed herein are IL-17 antagonists (e.g., IL-17 antibody or antigen-binding fragment thereof, e.g., secukinumab) for use in treating a patient having nr-axSpA, wherein the IL-17 antagonist (e.g., IL-17 antibody or antigen-binding fragment thereof, e.g., secukinumab) binds to an epitope of an IL-17 homodimer having two mature IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain, wherein the IL-17 antibody or antigen-binding fragment thereof has a K_{D} of about 100-200 pM, and wherein the IL-17 antibody or antigen-binding fragment thereof has an in vivo half-life of about 4 weeks.

Additionally disclosed herein are IL-17 antagonists (e.g., IL-17 antibody or antigen-binding fragment thereof, e.g., secukinumab) for use in inhibiting the progression of structural damage in an nr-axSpA patient, wherein the IL-17 antagonist (e.g., IL-17 antibody or antigen-binding fragment thereof, e.g., secukinumab) binds to an epitope of an IL-17 homodimer having two mature IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain, wherein the IL-17 antibody or antigen-binding fragment thereof has a K_{D} of about 100-200 pM, and wherein the IL-17 antibody or antigen-binding fragment thereof has an in vivo half-life of about 4 weeks.

Additionally disclosed herein are IL-17 antagonists (e.g., IL-17 antibody or antigen-binding fragment thereof, e.g., secukinumab) for use in the manufacture of a medicament for treating a patient having nr-axSpA, wherein the IL-17 antagonist (e.g., IL-17 antibody or antigen-binding fragment thereof, e.g., secukinumab) binds to an epitope of an IL-17 homodimer having two mature IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain, wherein the IL-17 antibody or antigen-binding fragment thereof has a K_{D} of about 100-200 pM, and wherein the IL-17 antibody or antigen-binding fragment thereof has an in vivo half-life of about 4 weeks.

Additionally disclosed herein are IL-17 antagonists (e.g., IL-17 antibody or antigen-binding fragment thereof, e.g., secukinumab) for use in the manufacture of a medicament for inhibiting the progression of structural damage in an nr-axSpA patient, wherein the IL-17 antagonist (e.g., IL-17 antibody or antigen-binding fragment thereof, e.g., secukinumab) binds to an epitope of an IL-17 homodimer having two mature IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain, wherein the IL-17 antibody or antigen-binding fragment thereof has a K_{D} of about 100-200 pM, and wherein the IL-17 antibody or antigen-binding fragment thereof has an in vivo half-life of about 4 weeks.

Additionally disclosed herein are IL-17 antagonists (e.g., IL-17 antibody or antigen-binding fragment thereof, e.g., secukinumab) for use in the manufacture of a medicament for treating a patient having nr-axSpA, wherein the medicament is formulated to comprise containers, each container having a sufficient amount of the IL-17 antagonist (e.g., IL-17 antibody or antigen-binding fragment thereof, e.g., secukinumab) to allow subcutaneous delivery of at least about 75 mg-about 300 mg (e.g., about 75 mg, about 150 mg, about 300 mg) of the IL-17 antagonist (e.g., IL-17 antibody or antigen-binding fragment thereof, e.g., secukinumab) per unit dose, and further wherein the IL-17 antagonist (e.g., IL-17 antibody or antigen-binding fragment thereof, e.g., secukinumab) binds to an epitope of an IL-17 homodimer having two mature IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain, wherein the IL-17 antibody or antigen-binding fragment thereof has a K_{D} of about 100-200 pM, and wherein the IL-17 antibody or antigen-binding fragment thereof has an in vivo half-life of about 4 weeks.

Additionally disclosed herein are IL-17 antagonists (e.g., IL-17 antibody or antigen-binding fragment thereof, e.g., secukinumab) for use in the manufacture of a medicament for inhibiting the progression of structural damage in an nr-axSpA patient, wherein the medicament is formulated to comprise containers, each container having a sufficient amount of the IL-17 antagonist (e.g., IL-17 antibody or antigen-binding fragment thereof, e.g., secukinumab) to allow subcutaneous delivery of at least about 75 mg-about 300 mg (e.g., about 75 mg, about 150 mg, about 300 mg) IL-17 antagonist (e.g., IL-17 antibody or antigen-binding fragment thereof, e.g., secukinumab) per unit dose, and further wherein the IL-17 antagonist (e.g., IL-17 antibody or antigen-binding fragment thereof, e.g., secukinumab) binds to an epitope of an IL-17 homodimer having two mature IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain, wherein the IL-17 antibody or antigen-binding fragment thereof has a K_{D} of about 100-200 pM, and wherein the IL-17 antibody or antigen-binding fragment thereof has an in vivo half-life of about 4 weeks.

As used herein, the phrase “formulated at a dosage to allow [route of administration] delivery of [a designated dose]” is used to mean that a given pharmaceutical composition can be used to provide a desired dose of an IL-17 antagonist, e.g., an IL-17 antibody, e.g., secukinumab, via a designated route of administration (e.g., SC or IV). As an example, if a desired subcutaneous dose is 300 mg, then a clinician may use 2 ml of an IL-17 antibody formulation having a concentration of 150 mg/ml, 1 ml of an IL-17 antibody formulation having a concentration of 300 mg/ml, 0.5 ml of an IL-17 antibody formulation having a concentration of 600 mg/ml, etc. In each such case, these IL-17 antibody formulations are at a concentration high enough to allow subcutaneous delivery of the IL-17 antibody. Subcutaneous delivery typically requires delivery of volumes of less than about 2 ml, preferably a volume of about 1 ml or less. Preferred formulations are liquid pharmaceutical compositions comprising about 25 mg/mL to about 150 mg/mL secukinumab, about 10 mM to about 30 mM histidine pH 5.8, about 200 mM to about 225 mM trehalose, about 0.02% polysorbate 80, and about 2.5 mM to about 20 mM methionine.

As used herein, the phrase “container having a sufficient amount of the IL-17 antagonist to allow delivery of [a designated dose]” is used to mean that a given container (e.g., vial, pen, syringe) has disposed therein a volume of an IL-17 antagonist (e.g., as part of a pharmaceutical composition) that can be used to provide a desired dose. As an example, if a desired dose is 150 mg, then a clinician may use 2 ml from a container that contains an IL-17 antibody formulation with a concentration of 75 mg/ml, 1 ml from a container that contains an IL-17 antibody formulation with a concentration of 150 mg/ml, 0.5 ml from a container contains an IL-17 antibody formulation with a concentration of 300 mg/ml, etc. In each such case, these containers have a sufficient amount of the IL-17 antagonist to allow delivery of the desired 150 mg dose.

In some embodiments of the disclosed uses, methods, and kits, the patient moderate to severe nr-axSpA. In some embodiments of the disclosed uses, methods, and kits, the patient has severe nr-axSpA. In some embodiments of the disclosed uses, methods, and kits, the patient has active nr-axSpA.

In some embodiments of the disclosed uses, methods, and kits, the patient has active nr-axSpA as assessed by total BASDAI≥4. In some embodiments of the disclosed uses, methods, and kits, the patient has total BASDAI≥4 cm (0-10 cm) at baseline, spinal pain as measured by BASDAI question number 2≥4 cm (0-10 cm) at baseline, and total back pain as measured by VAS≥40 mm (0-100 mm) at baseline.

In some embodiments of the disclosed uses, methods, and kits, the patient has nr-axSpA according to the ASAS axSpA criteria. In some embodiments of the disclosed uses, methods, and kits, a) the patient has had inflammatory back pain for at least three, preferably at least six months, b) the onset of the inflammatory back pain of a) occurred before the patient was 45 years old, and c) the patient has MRI evidence of sacroiliac joint inflammation and has at least one SpA feature or the patient is HLA-B27 positive and has at least two SpA features.

In some embodiments of the disclosed uses, methods, and kits, the patient has objective signs of inflammation as indicated by elevated C-reactive protein (CRP) and/or magnetic resonance imaging (MRI) evidence of sacroiliac joint inflammation. In some embodiments of the disclosed uses, methods, and kits, the patient has objective signs of inflammation as indicated by MRI evidence of sacroiliac joint inflammation determined according to the Berlin sacroiliac joint (SIJ) scoring method. In some embodiments of the disclosed uses, methods, and kits, the patient has objective signs of inflammation as indicated by MRI evidence of inflammation of the spine.

In some embodiments of the disclosed uses, methods, and kits, the patient does not satisfy the radiological criterion according to the modified New York diagnostic criteria for ankylosing spondylitis.

In some embodiments of the disclosed uses, methods, and kits, the patient had previously failed to respond to, or had an inadequate response to, treatment with a nonsteroidal anti-inflammatory drug (NSAID). In some embodiments of the disclosed uses, methods, and kits, the patient had previously failed to respond to, or had an inadequate response to, treatment with a TNF-alpha inhibitor (TNF-IR). In some embodiments of the disclosed uses, methods, and kits, the patient had not previously been treated with a TNF-alpha antagonist (TNF-naïve).

In some embodiments of the disclosed uses, methods, and kits, the patient is additionally administered comprising administering cyclosporine, hydroxychloroquine, methotrexate, an NSAID, sulfasalazine, leflunomide, prednisolone, prednisone, or methylprednisolone to the patient.

In some embodiments of the disclosed uses, methods, and kits, the patient is administered about 75 mg-about 300 mg of the IL-17 antibody or antigen-binding fragment thereof by subcutaneous injection at weeks 0, 1, 2 and 3, followed by once monthly dosing starting at week 4. In some embodiments of the disclosed uses, methods, and kits, the patient is administered 150 mg of the IL-17 antibody or antigen-binding fragment thereof by subcutaneous injection at weeks 0, 1, 2 and 3, followed by once monthly dosing starting at week 4.

In some embodiments of the disclosed uses, methods, and kits, the IL-17 antibody or antigen-binding fragment thereof comprises: i) an immunoglobulin heavy chain variable domain (V_{H}) comprising the amino acid sequence set forth as SEQ ID NO:8; ii) an immunoglobulin light chain variable domain (V_{L}) comprising the amino acid sequence set forth as SEQ ID NO: 10; iii) an immunoglobulin V_{H} domain comprising the amino acid sequence set forth as SEQ ID NO:8 and an immunoglobulin V_{L} domain comprising the amino acid sequence set forth as SEQ ID NO: 10; iv) an immunoglobulin V_{H} domain comprising the hypervariable regions set forth as SEQ ID NO: 1, SEQ ID NO:2, and SEQ ID NO:3; v) an immunoglobulin V_{L} domain comprising the hypervariable regions set forth as SEQ ID NO:4, SEQ ID NO:5 and SEQ ID NO:6, vi) an immunoglobulin V_{H} domain comprising the hypervariable regions set forth as SEQ ID NO: 11, SEQ ID NO:12 and SEQ ID NO:13; vii) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO:5 and SEQ ID NO:6; viii) an immunoglobulin V_{H} domain comprising the hypervariable regions set forth as SEQ ID NO: 11, SEQ ID NO: 12 and SEQ ID NO: 13 and an immunoglobulin V_{L} domain comprising the hypervariable regions set forth as SEQ ID NO:4, SEQ ID NO:5 and SEQ ID NO:6; ix) an immunoglobulin light chain comprising the amino acid sequence set forth as SEQ ID NO: 14; x) an immunoglobulin heavy chain comprising the amino acid sequence set forth as SEQ ID NO: 15; or xi) an immunoglobulin light chain comprising the amino acid sequence set forth as SEQ ID NO:14 and an immunoglobulin heavy chain comprising the amino acid sequence set forth as SEQ ID NO: 15. In some embodiments of the disclosed uses, methods, and kits, the IL-17 antibody or antigen-binding fragment thereof is secukinumab.

Disclosed herein are also methods of treating a patient having severe active axial spondyloarthritis (axSpA) without radiographic evidence of ankylosing spondylitis, but with objective signs of inflammation as indicated by CRP and/or MRI, comprising administering the patient about 150 mg of secukinumab by subcutaneous injection at weeks 0, 1, 2 and 3, followed by once monthly dosing starting at week 4.

Disclosed herein are also methods of treating a patient having severe axSpA without radiographic evidence of AS, but with objective signs of inflammation by elevated CRP and/or MRI, wherein the patient previously had an inadequate response to treatment with an NSAID, comprising administering the patient about 150 mg of secukinumab by subcutaneous injection at weeks 0, 1, 2 and 3, followed by once monthly dosing starting at week 4.

Disclosed herein are also methods of treating a patient having severe axSpA without radiographic evidence of AS, but with objective signs of inflammation by elevated CRP and/or MRI, wherein the patient previously had an inadequate response to, or was intolerant to treatment with an NSAID, comprising administering the patient about 150 mg of secukinumab by subcutaneous injection at weeks 0, 1, 2 and 3, followed by once monthly dosing starting at week 4.

Disclosed herein are also methods of treating a patient having severe axSpA without radiographic evidence of AS, but with objective signs of inflammation by elevated CRP and/or MRI, wherein the patient previously failed to respond to, or had an inadequate response to, treatment with a TNF-alpha inhibitor, comprising administering the patient about 150 mg of secukinumab by subcutaneous injection at weeks 0, 1, 2 and 3, followed by once monthly dosing starting at week 4.

Disclosed herein are also methods of treating a patient having severe axSpA without radiographic evidence of AS, but with objective signs of inflammation by elevated CRP and/or MRI, wherein the patient previously had an inadequate response to treatment with a TNF-alpha inhibitor, comprising administering the patient about 150 mg of secukinumab by subcutaneous injection at weeks 0, 1, 2 and 3, followed by once monthly dosing starting at week 4.

Disclosed herein are also methods of treating a patient having severe axSpA, wherein the patient has not previously been treated with a TNF-alpha antagonist (TNF naïve), comprising administering the patient about 150 mg of secukinumab by subcutaneous injection at weeks 0, 1, 2 and 3, followed by once monthly dosing starting at week 4.

Disclosed herein are also methods of treating a patient having severe axSpA without radiographic evidence of AS, but with objective signs of inflammation by elevated CRP and/or MRI, wherein the patient has not previously been treated with a TNF-alpha antagonist (TNF naïve), comprising administering the patient about 150 mg of secukinumab by subcutaneous injection at weeks 0, 1, 2 and 3, followed by once monthly dosing starting at week 4.

Disclosed herein are also methods of treating a patient having severe axSpA without radiographic evidence of AS, but with objective signs of inflammation by elevated CRP and/or MRI, wherein the patient has not previously been treated with a TNF-alpha antagonist (TNF naïve), comprising administering the patient about 150 mg of secukinumab by subcutaneous injection at weeks 0, 1, 2 and 3, followed by once monthly dosing starting at week 4.

### Kits

The disclosure also encompasses kits for preventing structural damage (e.g., bone and joint) in an nr-axSpA patient. Such kits comprise an IL-17 antagonist, e.g., IL-17 binding molecule (e.g., IL-17 antibody or antigen-binding fragment thereof, e.g., secukinumab) or IL-17 receptor binding molecule (e.g., IL-17 antibody or antigen-binding fragment thereof) (e.g., in liquid or lyophilized form) or a pharmaceutical composition comprising the IL-17 antagonist (described supra). Additionally, such kits may comprise means for administering the IL-17 antagonist (e.g., an autoinjector, a syringe and vial, a prefilled syringe, a prefilled pen) and instructions for use. These kits may contain additional therapeutic agents (described supra) for treating nr-axSpA, e.g., for delivery in combination with the enclosed IL-17 antagonist, e.g., IL-17 binding molecule, e.g., IL-17 antibody, e.g., secukinumab. Such kits may also comprise instructions for administration of the IL-17 antagonist (e.g., IL-17 antibody, e.g., secukinumab) to treat the nr-axSpA patient and/or to inhibit the progression of structural damage in the nr-axSpA patient (e.g., TNF-naïve and/or TNF-IR nr-axSpA patients, NSAID failure nr-axSpA patients, etc.). Such instructions may provide the dose (e.g., 10 mg/kg, 75 mg, 150 mg, 300 mg), route of administration (e.g., IV, SC), and dosing regimen (e.g., about 10 mg/kg given IV, every other week during weeks 0, 2, and 4, and thereafter at about 75 mg, about 150 mg, or about 300 mg given SC monthly, beginning during week 8; about 75 mg, about 150 mg, or about 300 mg given SC weekly during week 0, 1, 2, and 3 and thereafter at about 75 mg, about 150 mg, or about 300 mg given SC monthly, beginning during week 4; about 75 mg, about 150 mg, or about 300 mg given SC monthly, etc.) for use with the enclosed IL-17 antagonist, e.g., IL-17 binding molecule, e.g., IL-17 antibody, e.g., secukinumab.

The phrase “means for administering” is used to indicate any available implement for systemically administering a drug to a patient, including, but not limited to, a pre-filled syringe, a vial and syringe, an injection pen, an autoinjector, an IV drip and bag, a pump, etc. With such items, a patient may self-administer the drug (i.e., administer the drug without the assistance of a physician) or a medical practitioner may administer the drug.

Disclosed herein are kits for use in treating a patient having nr-axSpA and/or inhibiting the progression of structural damage in an nr-axSpA patient, comprising an IL-17 antagonist (e.g., IL-17 binding molecule, e.g., IL-17 antibody or antigen-binding fragment thereof, e.g., secukinumab). In some embodiments, the kit further comprises means for administering the IL-17 antagonist to the patient. In some embodiments, the kit further comprises instructions for administration of the IL-17 antagonist, wherein the instructions indicate that the IL-17 antagonist (e.g., IL-17 binding molecule, e.g., IL-17 antibody or antigen-binding fragment thereof, e.g., secukinumab) is to be administered to the patient (e.g., TNF naïve and/or TNF experienced) intravenously (IV) at about 10 mg/kg every other week during week 0, 2, and 4 and thereafter is to be administered to the patient subcutaneously (SC) at about 75 mg-about 300 mg (e.g., about 75 mg, about 150 mg, or about 300 mg) monthly, beginning during week 8. In some embodiments, the kit further comprises instructions for administration of the IL-17 antagonist, wherein the instructions indicate that the IL-17 antagonist (e.g., IL-17 binding molecule, e.g., IL-17 antibody or antigen-binding fragment thereof, e.g., secukinumab) is to be administered to the patient SC with or without a loading regimen, e.g., at about 75 mg-about 300 mg (e.g., about 75 mg, about 150 mg, or about 300 mg) weekly during weeks 0, 1, 2, and 3, and thereafter SCat about 75 mg-about 300 mg (e.g., about 75 mg, about 150 mg, or about 300 mg) monthly, beginning during week 4; or about 75 mg-about 300 mg (e.g., about 75 mg, about 150 mg, or about 300 mg) every 4 weeks (monthly). In some embodiments, the instructions will provide for dose escalation (e.g., from a dose of about 75 mg to a higher dose of about 150 mg or about 300 mg as needed, to be determined by a physician).

In some embodiments, the kit further comprises instructions for administration of the IL-17 antagonist, wherein the instructions indicate that the IL-17 antagonist (e.g., IL-17 binding molecule, e.g., IL-17 antibody or antigen-binding fragment thereof, e.g., secukinumab) is to be administered to the patient SC at about 75 mg-about 300 mg (e.g., about 75 mg, about 150 mg, or about 300 mg) weekly during weeks 0, 1, 2, and 3, and thereafter is to be administered to the patient SC at about 75 mg-about 300 mg (e.g., about 75 mg, about 150 mg, or about 300 mg) monthly, beginning during week 4; or about 75 mg-about 300 mg (e.g., about 75 mg, about 150 mg, or about 300 mg) every 4 weeks (monthly). In some embodiments, the instructions will provide for dose escalation (e.g., from a dose of about 75 mg to a higher dose of about 150 mg or about 300 mg as needed, to be determined by a physician).

### General

In preferred embodiments of the disclosed methods, treatments, medicaments, regimens, uses and kits, the IL-17 antagonist is an IL-17 binding molecule. In preferred embodiments, the IL-17 binding molecule is an IL-17 antibody or antigen-binding fragment thereof. In some embodiments of the disclosed methods, treatments, regimens, uses and kits, the IL-17 antibody or antigen-binding fragment thereof is selected from the group consisting of: a) an IL-17 antibody or antigen-binding fragment thereof that binds to an epitope of IL-17 comprising Leu74, Tyr85, His86, Met87, Asn88, Val124, Thr125, Pro126. Ile127. Val128, His129: b) an IL-17 antibody or antigen-binding fragment thereof that binds to an epitope of IL-17 comprising Tyr43, Tyr44, Arg46, Ala79, Asp80; c) an IL-17 antibody or antigen-binding fragment thereof that binds to an epitope of an IL-17 homodimer having two mature IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain; d) an IL-17 antibody or antigen-binding fragment thereof that binds to an epitope of an IL-17 homodimer having two mature IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain, wherein the 1-17 binding molecule has a K_{D} of about 100-200 pM, and wherein the IL-17 binding molecule has an in vivo half-life of about 23 to about 35 days; and e) an IL-17 antibody or antigen-binding fragment thereof comprising: i) an immunoglobulin heavy chain variable domain (V_{H}) comprising the amino acid sequence set forth as SEQ ID NO:8; ii) an immunoglobulin light chain variable domain (V_{L}) comprising the amino acid sequence set forth as SEQ ID NO: 10; iii) an immunoglobulin V_{H} domain comprising the amino acid sequence set forth as SEQ ID NO:8 and an immunoglobulin V_{L} domain comprising the amino acid sequence set forth as SEQ ID NO: 10; iv) an immunoglobulin V_{H} domain comprising the hypervariable regions set forth as SEQ ID NO: 1, SEQ ID NO:2, and SEQ ID NO:3; v) an immunoglobulin V_{L} domain comprising the hypervariable regions set forth as SEQ ID NO:4, SEQ ID NO:5 and SEQ ID NO:6; vi) an immunoglobulin V_{H} domain comprising the hypervariable regions set forth as SEQ ID NO: 11, SEQ ID NO:12 and SEQ ID NO:13; vii) an immunoglobulin V_{H} domain comprising the hypervariable regions set forth as SEQ ID NO: 1, SEQ ID NO:2, and SEQ ID NO:3 and an immunoglobulin V_{L} domain comprising the hypervariable regions set forth as SEQ ID NO:4, SEQ ID NO:5 and SEQ ID NO:6; viii) an immunoglobulin V_{H} domain comprising the hypervariable regions set forth as SEQ ID NO: 11, SEQ ID NO:12 and SEQ ID NO:13 and an immunoglobulin V_{L} domain comprising the hypervariable regions set forth as SEQ ID NO:4, SEQ ID NO:5 and SEQ ID NO:6; ix) an immunoglobulin light chain comprising the amino acid sequence set forth as SEQ ID NO: 14; x) an immunoglobulin heavy chain comprising the amino acid sequence set forth as SEQ ID NO: 15; or xi) an immunoglobulin light chain comprising the amino acid sequence set forth as SEQ ID NO: 14 and an immunoglobulin heavy chain comprising the amino acid sequence set forth as SEQ ID NO:15.

In some embodiments of the disclosed methods, the IL-17 antibody or antigen-binding fragment thereof is a human antibody of the IgG₁ isotype. In some embodiments of the disclosed methods, the antibody or antigen-binding fragment thereof is secukinumab.

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference. The following Examples are presented in order to more fully illustrate the preferred embodiments of the disclosure. These examples should in no way be construed as limiting the scope of the disclosed patient matter, as defined by the appended claims.

### EXAMPLES

### Example 1: Proof of Concept AS Trial CAIN457A2209

### Example 1.1—Study Design CAIN457A2209

This was a two-part multi-center proof of concept study of multiple 10 mg/kg, 1.0 mg/kg and 0.1 mg/kg doses of secukinumab (2 infusions given 3 weeks apart) for the treatment of patients with a diagnosis of moderate to severe AS with or without previous TNF antagonist therapy. In Part 1, 30 patients received either secukinumab 10 mg/kg or placebo in a 4:1 ratio. In Part 2, a further 30 patients received either secukinumab 0.1 mg/kg, 1.0 mg/kg or 10 mg/kg in a 2:2:1 ratio. The study consisted of a screening period of 28 days; a treatment period of 3 weeks, and a follow-up period of 25 weeks. Subjects who met the inclusion/exclusion criteria at screening underwent baseline evaluations, including the ASAS core set domains (1-6) (Zochling et al (2006) Ann Rheum Dis 65:442-452), BASMI score, BASDAI score and physician global assessment. The primary end point for this trial was the proportion of patients achieving the ASAS20 response at Week 6.

Patients with moderate to severe AS fulfilling the modified New York criteria for a diagnosis of AS and whose disease was not controlled on NSAIDs (on at least one NSAID over a period of at least 3 months at the maximum tolerated dose) were randomized to receive 2×10 mg/kg AIN457 or placebo. Minimum disease activity for inclusion of patients was assessed based on the ASAS core set domains: total back pain or nocturnal back pain score ≥40 (0-100 mm VAS) despite concurrent NSAID use, PLUS a total BASDAI score ≥4. Concomitant use of stable doses of methotrexate (MTX), sulfasalazine (SSZ) and low-dose corticosteroids was allowed as defined in the inclusion/exclusion criteria. Immunosuppressive agents other than MTX, SSZ and systemic low-dose corticosteroids required a 1-month wash-out period prior to baseline.

Efficacy evaluations were based on the ASAS core assessment criteria, consisting of the following assessment domains: (1) patient global assessment (PGA), (2) inflammatory back pain (mean of responses to questions #5 and #6 or the Bath Ankylosing Spondylitis Disease Activity Index [BASDAI]), (3) Bath Ankylosing Spondylitis Functional Index (BASFI), (4) inflammatory back pain (measured by total back pain or nocturnal back pain on a 0-100 mm VAS). Secondary objectives included magnetic resonance imaging (MRI) studies of the spine using a scoring system for quantification of AS-related pathologies, to investigate whether these changes are affected by treatment with secukinumab. Exploratory goals of the study were to define biomarker profiles using genetic, mRNA expression profiling, flow cytometry, and serum protein assessments in patients with moderate to severe AS, and to determine whether treatment with secukinumab affects these biomarkers.

Thirty (30) patients were randomized in a 4:1 ratio to receive two i.v infusions of either secukinumab (AIN457) 10 mg/kg IV or placebo IV given 3 weeks apart (Day 1 and Day 22). Patients were followed for safety up to week 28. A Bayesian analysis of the Week 6 ASAS20 response rates of AIN457 and placebo was performed. The prior distributions for the response rates were specified as Beta distributions and the binomial distribution was assumed for the observed number of responders in each group. The predictive distribution of the placebo response rate from a meta-analysis of 8 randomized, placebo-controlled trials of anti-TNF-alpha treatment in AS was used as the prior distribution for the placebo response rate. This prior distribution was equivalent to observing 11 out of 43 responders (i.e., a response rate of 26%). A weak prior distribution was used for the active response rate (equivalent to observing 0.5 out of 1.5 responders). Sagittal MR images of the spine were performed including T1- and short tau inversion recovery (STIR) sequences at baseline, Week 6 and Week 28. Images were analyzed by an independent reader, who was blinded to treatment allocation and chronology of images, using the “Berlin modification” of the AS spinal MRI (ASspiMRI-a) scoring system. Wilcoxon signed-rank test was used for the evaluation of changes between baseline and follow-up in each treatment arm.

### ASAS (Assessment in SpondyloArthritis International Society) Criteria

The ASAS (Assessment in SpondyloArthritis International Society) assessment criteria (1-6) consists of the following assessment domains: (1) Patient global assessment of disease activity, assessed on a 100 mm visual analogue scale (VAS); (2) Pain, assessed by the VAS pain score (0-100 scale) or NRS (0-10); (3) Physical function, assessed by BASFI score (0-100 scale); (4) Inflammation, assessed by the mean of the two morning stiffness-related BASDAI questions #5 and #6 on a 10 point scale or 100 mm VAS scores; (5) Bath Ankylosing Spondylitis Metrology Index (BASMI); scores (cervical rotation, chest expansion, lumbar lateral flexion, modified Schober index, occiput-to-wall distance); (6) C-reactive protein (acute phase reactant).

ASAS20 Responder Definition

A subject is defined as an ASAS20 responder if, and only if, both of the following conditions hold:

1. they have a ≥20% improvement and an absolute improvement ≥1 unit in ≥3 of the following 4 core ASAS domains: Patient Global Assessment (measured on a VAS from 0-ain (measured as total back pain or nocturnal back pain on a VAS from 0-100 mm); Physical function (as measured by the BASFI, 0-10); Inflammation (as measured by the mean of the two morning stiffness related questions #5 and #6 from the BASDAI, 0-10);

2. they have no deterioration in the potential remaining domain (deterioration is defined as ≥20% worsening and an absolute worsening of ≥1 unit from baseline).

ASAS40 Responder Definition

A subject is defined as an ASAS40 responder if, and only if, both of the following conditions hold:

1. they have ≥40% improvement and an absolute improvement ≥2 units in 3 of the following 4 domains: Patient Global Assessment (measured on a VAS from 0-100 mm); Back pain (measured as total back pain or nocturnal back pain on a VAS from 0-100 mm); Physical function (as measured by the BASFI, 0-10); Inflammation (as measured by the mean of the two morning stiffness related questions #5 and #6 from the BASDAI, 0-10);

2. they have no worsening at all in the potential remaining domain of >0%° or >0 unit) from baseline.

ASAS 5/6 Responder Definition

A subject is defined as an ASAS 5/6 responder if, and only if, they have ≥20% improvement in five out of the following six ASAS domains: Patient Global Assessment (measured on a VAS from 0-100 mm); Back pain (measured as total back pain or nocturnal back pain on a VAS from 0-100 mm); Physical function (as measured by the BASFI, 0-10); Inflammation (as measured by the mean of the two morning stiffness related questions #5 and #6 from the BASDAI, 0-10); Bath Ankylosing Spondylitis Metrology Index (BASMI); scores (cervical rotation, chest expansion, lumbar lateral flexion, modified Schober index, occiput-to-wall distance); (6) C-reactive protein (acute phase reactant).

ASAS Partial Remission Definition

A subject is defined as achieving partial remission if, and only if, they have a value of <2 units in each of the following 4 core ASAS domains: Patient Global Assessment (measured on a VAS from 0-100 mm); Back pain (measured as total back pain or nocturnal back pain on a VAS from 0-100 mm); Physical function (as measured by the BASFI, 0-10); Inflammation (as measured by the mean of the two morning stiffness related questions #5 and #6 from the BASDAI, 0-10).

Bath Ankylosing Spondylitis Functional Index (BASFI)

The BASFI is a set of 10 questions designed to determine the degree of functional limitation in those patients with AS. The ten questions were chosen with a major input from patients with AS. The first 8 questions consider activities related to functional anatomy. The final 2 questions assess the patients' ability to cope with everyday life. A 10 cm visual analog scale is used to answer the questions. The mean of the ten scales gives the BASFI score—a value between 0 and 10.

Bath Ankylosing Spondylitis Disease Activity Index (BASDAI)

The BASDAI consists of a 0-10 scale (0 being no problem and 10 being the worst problem), which is used to answer 6 questions pertaining to the 5 major symptoms of AS: 1. Fatigue; 2. Spinal pain; 3. Joint pain/swelling; 4. Areas of localized tenderness (called enthesitis, or inflammation of tendons and ligaments); 5. Morning stiffness duration; 6. Morning stiffness severity. To give each symptom equal weighting, the mean (average) of the two scores relating to morning stiffness is added to the scores of the other 4 questions. The resulting 0 to 50 score is divided by 5 to give a final 0-10 BASDAI score. BASDAI scores of 4 or greater suggest suboptimal control of disease, and patients with scores of 4 or greater are usually good candidates for either a change in their medical therapy or for enrollment in clinical trials evaluating new drug therapies directed at AS.

Patient's Global Assessment of Disease Activity

The patient's global assessment of disease activity will be performed using a 100 mm VAS ranging from no disease activity to maximal disease activity in response to the question, “Considering all the ways your arthritis affects you, draw a line on the scale for how well you are doing”. At the investigator's site, the distance in mm from the left edge of the scale was measured and the value was entered on the eCRF.

Patient's Assessment of Pain Intensity

The patient's assessment of back pain will be performed using a 100 mm VAS ranging from no pain to unbearable pain, as assessed separately for total back pain or nocturnal back pain. At the investigator's site the distance in mm from the left edge of the scale will be measured and the value will be entered on the eCRF.

Bath Ankylosing Spondylitis Metrology Index (BASMI)

The BASMI is a validated instrument that uses the minimum number of clinically appropriate measurements that assess accurately axial status, with the goal to define clinically significant changes in spinal movement. Parameters include 1. cervical rotation; 2. tragus to wall distance; 3. lumbar side flexion; 4. modified Schober's; 5. intermalleolar distance. Two additional parameters are also assessed: 6. chest expansion and 7. occiput-to-wall distance.

Maastricht Ankylosing Spondylitis Enthesitis Score (MASES)

The Maastricht Ankylosing Spondylitis Enthesitis Score (MASES) was developed from the Mander index, and includes assessments of 13 sites. Enthesitis sites included in the MASES index are: 1st costochondral, 7^{th} costochondral, posterior superior iliac spine, anterior superior iliac spine, iliac crest (all above will be assessed bilaterally), 5th lumbar spinous process, proximal Achilles (bilateral).

Leeds Enthesis Index (LEI)

LEI is a validated enthesis index that uses only 6 sites for evaluation of enthesis: lateral epicondyle humerus L+R, proximal achilles L+R and lateral condyle femur. While LEI demonstrated substantial to excellent agreement with other scores in the indication of psoriatic arthritis, LEI demonstrated a lower degree of agreement with MASES in ankylosing spondylitis and might thus yield additional information in this indication.

MRI

Magnetic resonance imaging (MRI) of the spine was performed using a scoring system for quantification of AS-related pathologies, to investigate whether these changes were affected by treatment with secukinumab. MRIs were acquired locally at the clinical sites, and images were transmitted, quality controlled, de-identified (if necessary) and analyzed centrally (blinded review). MRI scans were collected at baseline (preferably within 2 weeks prior to first treatment) and at Week 6 (±1 week) and Week 28 (±1 week). MRI scans included pre- and post-intravenous gadolinium contrast enhanced MRI for evaluating inflammation and fat-saturating techniques such as short tau inversion recovery (STIR) to monitor bone marrow edema. The analysis method is the ‘Berlin modification of ASspiMRI-a’ (Lukas C et al (2007) J Rheumatol; 34(4):862-70 and Rudwaleit et al (2005) [abstract] Arthritis Rheum 50: S211), which scores inflammatory changes in nearly the entire vertebral column (C2-S1).

### Example 1.2—Secukinumab Shows Good Safety and Efficacy in the Treatment of Active Ankylosing Spondylitis

Demographics and baseline characteristics were comparable between groups. Mean (SD) BASDAI at baseline was 7.1 (1.4) for secukinumab-treated patients and 7.2 (1.8) for placebo-treated patients. Three patients on placebo and 2 patients on secukinumab discontinued the study prior to the primary endpoint, mostly due to unsatisfactory therapeutic effect. Efficacy data from 1 patient was not available due to a protocol violation after randomization. At Week 6, 14/23 secukinumab-treated patients who entered efficacy analysis achieved ASAS20 responses versus 1/6 placebo treated patients (61% vs 17%, probability of positive-treatment difference=99.8%, 95% credible interval 11.5%, 56.3%) (Table 3).

TABLE 3
Week 6 results for trial CAIN457A2209

| | | | | 95% | |
|---|---|---|---|---|---|
| | # of Responders | Response | Difference | credible | Probability |
| | (%) | rate | (vs. placebo) | interval | (Drug > Pbo) |
| AIN457 | 14/23 (60.9%) | 59.2% | 34.7% | 11.5%, 56.3% | 99.8% |
| Placebo | 1/6 (16.7%) | 24.5% | | | |

ASAS40 and ASAS5/6 responses of secukinumab-treated patients were 30% and 35%, respectively, and mean (range) BASDAI change was −1.8 (−5.6 to 0.8). In a majority of the ASAS20 responders, secukinumab induced responses within a week of treatment. ASAS response rates were greatest at the primary endpoint at Week 6, and declined thereafter up to end of study at Week 28, consistent with the preliminary dose regimen of only two doses of 10 mg/kg IVIV given at Days 1 and 22, as chosen for this proof-of-concept study. Post-hoc analyses of subgroups showed superior response rates with TNF alpha antagonist naïve (TNF naïve) patients (11/13; 85%) compared to TNF alpha antagonist pre-exposed patients (3/10; 30%). The pharmacokinetic profile was comparable to secukinumab given for other indications.

The primary endpoint of this study was met, as secukinumab induced significantly higher ASAS20 responses than placebo at Week 6. No early safety signals were noted in this study population.

### Example 1.3—Secukinumab Reduces Spinal Inflammation in Patients with AS as Early as Week 6, as Detected by Magnetic Resonance Imaging

Magnetic resonance imaging (MRI) is considered gold standard for assessment of spinal inflammation in AS. We thus determined whether clinical effects observed after 2 infusions (10 mg/kg IV) of secukinumab coincide with reductions of bone marrow edema seen on MRI. Sagittal MRI of the spine was performed including T1- and short tau inversion recovery (STIR) sequences at baseline (BL), Week 6 and Week 28. Images were analyzed by an independent reader, who was blinded to treatment allocation and chronology of images, using the “Berlin modification” of the AS spinal MRI (ASspiMRI-a) scoring system. Changes between baseline and follow-up in each treatment arm were evaluated by Wilcoxon signed-rank test.

Twenty seven patients (22 secukinumab; 5 on placebo) had evaluable MRI images at baseline. Few patients (at Week 6: 2 secukinumab, 3 placebo; at Week 28: 6 secukinumab, 1 placebo) missed follow-up MRIs, mostly due to early discontinuation. MRI scores at baseline and changes at week 6 and Week 28 are shown in Table 4. MRI score improvements were seen as early as Week 6 and sustained up to week 28. Early improvements at Week 6 were especially noted in patients with higher baseline scores. Only minor changes were seen in patients on placebo.

TABLE 4
MRI scores and ASAS response at week 6 and 28 following treatment with secukinumab

| | Secukinumab 2 × 10 mg/kg | | | Placebo | | |
|---|---|---|---|---|---|---|
| | Baseline | Week 6 | Week 28* | Baseline | Week 6 | Week 28 |
| # of patients | 22 | 22 | 16 | 5 | 3 | 5 |
| ASAS20 | — | 14 | 6 | — | 1 | 1 |
| responders (n) | | | | | | |
| Mean Berlin | 9.2 ± 8.9 | 6.7 ± 6.6 | 5.7 ± 6.2 | 20.6 ± 20.2 | 21.0 ± 24.6 | 19.0 ± 19.3 |
| score ± SD | | | | | | |
| P-value (vs. | — | 0.10 | 0.16 | — | 0.50 | 0.25 |
| baseline) | | | | | | |

*Data from 6 patients who discontinued prior to week 28 (lack of response) were not analyzed.

The results of this exploratory study in patients with active AS suggests that after treatment with only 2 infusions of secukinumab, substantial reductions of spinal inflammation as detected by MRI occurred. MRI changes were seen as early as 6 weeks after start of treatment, and were maintained up to week 28. Results are consonant with MRI findings obtained in previous AS trials with TNF blockers. These results provide support that secukinumab may be a potential treatment for patients with active AS.

### Example 2: Phase III Clinical Trial CAIN457F2305 (MEASURE 1)

MEASURE 1 (NCT01358175) is a randomized, double-blind, placebo (PBO)-controlled trial that has demonstrated the efficacy and safety of secukinumab, a human anti-interleukin-17A monoclonal antibody, in subjects with ankylosing spondylitis (AS).

### Example 2.1—Results CAIN457F2305

Here, our objective is to evaluate the efficacy of intravenous loading and subcutaneous maintenance dosing of secukinumab on multiple endpoints.

371 adults with active AS were randomized to receive intravenous (IV) secukinumab 10 mg/kg (Week 0, 2, 4) followed by subcutaneous (SC) secukinumab 75 mg every 4 weeks (IV→75 SC), IV secukinumab 10 mg/kg (Week 0, 2, 4) followed by SC secukinumab 150 mg every 4 weeks (IV→150 SC), or placebo (PBO) on the same IV and SC schedules. PBO subjects were re-randomized to secukinumab 75 mg or 150 mg SC based on Assessment of Spondyloarthritis International Society (ASAS) 20 response at Week 16, with non-responders switched at Week 16 and responders at Week 24. Measures of disease activity (signs and symptoms) included Ankylosing Spondylitis Disease Activity Score (ASDAS)-C-reactive protein (ASDAS-CRP), ASDAS-erythrocyte sedimentation rate (ASDAS-ESR), and Bath Ankylosing Spondylitis Disease Activity Index (BASDAI). The effect of secukinumab on individual components of the ASAS response criteria (used to determine ASAS 20, ASAS 40, ASAS 5/6, and ASAS partial remission rates) are also reported: patient's global assessment of disease activity and inflammatory back pain, both assessed on a visual analogue scale (VAS), Bath Ankylosing Spondylitis Functional Index (BASFI), and spinal inflammation based on BASDAI questions 5 and 6.

The primary endpoint was met with both secukinumab groups in MEASURE 1. In MEASURE 1, ASAS20 response rates at week 16 were 60.8% with secukinumab IV→150 mg, 59.7% with secukinumab IV→75 mg, and 28.7% with placebo (P<0.001 for both comparisons versus placebo) (Table 5). Furthermore, all pre-defined secondary endpoints were met with both secukinumab groups in MEASURE 1 (Table 5). ASAS40 response rates at week 16 were 41.6%, 33.1%, and 13.1% in the secukinumab IV→0.150 mg, secukinumab IV→75 mg, and placebo groups, respectively (P<0.001 for both comparisons versus placebo) (Table 5). Improvements in patients treated with secukinumab were sustained through 52 weeks.

TABLE 5
Summary of Week 16 Efficacy Results in the MEASURE 1 Study
(Full Analysis Set).*

| | Secukinumab | Secukinumab | |
|---|---|---|---|
| | IV →150 mg | IV →75 mg | Placebo |
| Week 16 Data | (N = 125) | (N = 124) | (N = 122) |
| ASAS20 response^{†}, | 76 (60.8)^{‡} | 74 (59.7)^{‡} | 35 (28.7) |
| n (%) | | | |
| ASAS40 response^{‡}, | 52 (41.6)^{‡} | 41 (33.1)^{‡} | 16 (13.1) |
| n (%) | | | |
| hsCRP, post-baseline to | 0.40 ± 1.09^{‡} | 0.45 ± 1.09^{‡} | 0.97 ± 1.10 |
| baseline ratio | | | |
| (LSM ± SE) | | | |
| ASAS5/6 response^{§}, | 61 (48.8)^{‡} | 56 (45.2)^{‡} | 16 (13.1) |
| n (%) | | | |
| BASDAI^{¶}, mean change | −2.32 ± 0.17^{‡} | −2.34 ± 0.18^{‡} | −0.59 ± 0.18 |
| from baseline | | | |
| (LSM ± SE) | | | |
| SF-36 PCS, mean change | 5.57 ± 0.59^{‡} | 5.64 ± 0.60^{‡} | 0.96 ± 0.61 |
| from baseline | | | |
| (LSM ± SE) | | | |
| ASQoL^{\|\|}, mean change | −3.58 ± 0.42^{‡} | −3.61 ± 0.42^{‡} | −1.04 ± 0.44 |
| from baseline | | | |
| (LSM ± SE) | | | |
| ASAS partial | 19 (15.2)^{††} | 20 (16.1)^{††} | 4 (3.3) |
| remission**, n (%) | | | |

*Prespecified hierarchical testing strategy used to account for multiplicity of testing in overall study population. Missing data for binary variables imputed as non-response, Result from mixed-effect model repeated measures (MMRM).
^{†}≥20% improvement in 3 of 4 main ASAS response criteria, with no worsening of ≥20% in the fourth.
^{‡}P < 0.001 versus placebo.
^{‡}≥40% improvement in three of four main ASAS response criteria, with no worsening in the fourth.
^{§}≥20% improvement in five of the six ASAS response criteria.
^{¶}Scores range from 0 to 10, with 1 representing no problem and 10 the worst problem.
^{||}Scores range from 0 to 18, with 0 representing lowest severity and 18 highest severity.
**A score of <2 units (from 0 to 10) in each of the four core ASAS domains,
^{††}P < 0.01 versus placebo.
ASAS, Assessment of SpondyloArthritis International Society criteria;
ASQoL, Ankylosing Spondylitis Quality of Life;
BASDAI, Bath Ankylosing Spondylitis Disease Activity Index;
hsCRP, high-sensitivity C-reactive protein;
LSM, least squares mean;
SE, standard error.

### Example 2.2—Imaging CAIN457F2305 Weeks 16 and 52

Here our objective is to investigate the effect of secukinumab on objective signs of inflammation in the sacroiliac (SI) joints and spine at Weeks 16 and 52 using magnetic resonance imaging (MRI) in the MEASURE 1 study.

371 adults with active AS, despite maximally tolerated therapy with nonsteroidal anti-inflammatory drugs (NSAIDs), were randomized to secukinumab or placebo: IV secukinumab 10 mg/kg (weeks 0, 2, 4) followed by SC secukinumab 75 mg every 4 weeks (IV→75 SC); SCSC secukinumab 150 mg every 4 weeks (IV→150 SC); or PBO on the same schedules. MRI of the SI joints and spine were performed on a subset of 105 subjects with no prior exposure to therapies targeting tumor necrosis factor (anti-TNF-naïve). Assessments were completed at baseline, weeks Weeks 16, 52, and 104. MRI variables were assessed by the Berlin SI joint total edema score, MRI score for spinal activity (ASspi-MRI-a), and the Berlin spine score (derived from the ASspi-MRI-a results). Two experienced readers, blinded to treatment and visit, evaluate d all MRIs and their mean scores were used for the final analyses.

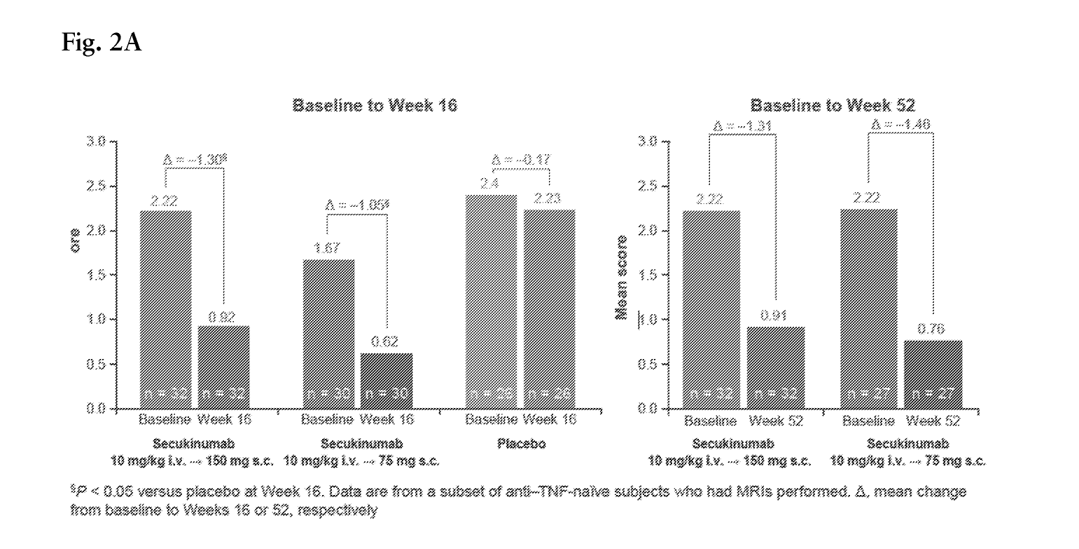

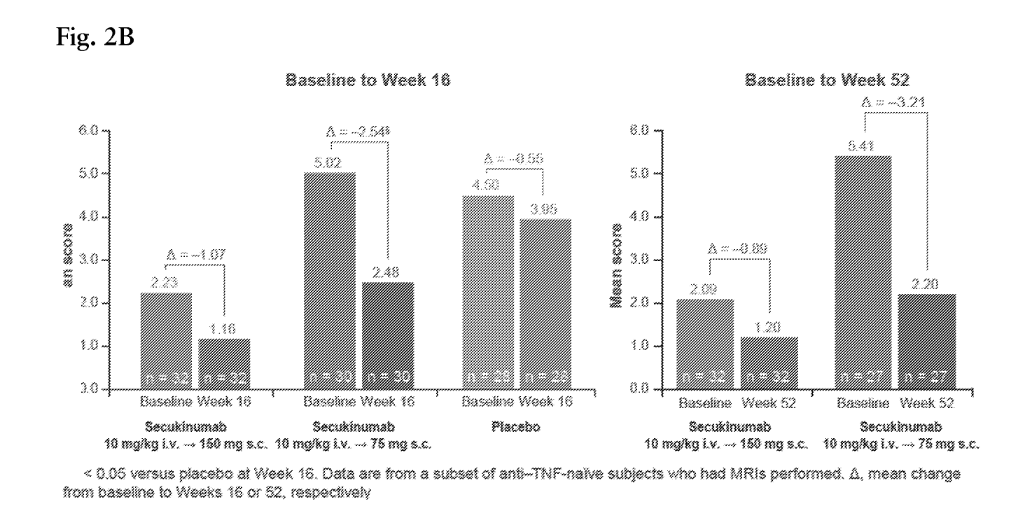

Mean baseline ASspi-MRI-a and Berlin spine scores were lower in the secukinumab IV→150 SC group than in the IV→75 SC and placebo groups (Table 6). At Week 16, improvements were shown in Berlin SI joint total edema score with secukinumab vs placebo (mean change from baseline: −1.30 and −1.05 vs −0.17 in secukinumab IV→150 SC and IV→75 SC vs placebo groups, respectively; P<0.01) (Table 6, FIG. 2A). Both secukinumab doses also resulted in greater mean percentage improvements from baseline in ASspi-MRI-a and Berlin spine scores vs placebo (Table 6, FIG. 2B). Improvements in all MRI measures with secukinumab were sustained through Week 52 (Table 7).

TABLE 6
MRI measurements at baseline, Week 16 and change from baseline
(MRI subset of TNF-alpha inhibitor naïve patients)

| | | | | | |
|---|---|---|---|---|---|
| | | | | Change from | p-value for |
| | | Baseline | Week 16 | baseline | comparison |
| MRI variable | n | (mean ± SD) | (mean ± SD) | (mean ± SD) [%] | vs. placebo |

Berlin sacroiliac joint total edema score

| | | | | | |
|---|---|---|---|---|---|
| IV→75 mg (N = 34) | 30 | 1.67 ± 2.551 | 0.62 ± 0.971 | −1.05 ± 2.090 [63%] | 0.0024 |
| IV→150 mg (N = 38) | 32 | 2.22 ± 3.377 | 0.92 ± 1.783 | −1.30 ± 2.170 [59%] | 0.0013 |
| Placebo (N = 33) | 26 | 2.40 ± 3.240 | 2.23 ± 3.238 | −0.17 ± 1.232 [7%] | |

Total ASspi-MRI-a score

| | | | | | |
|---|---|---|---|---|---|
| IV→75 mg (N = 34) | 30 | 6.37 ± 10.757 | 2.93 ± 6.403 | −3.43 ± 6.315 [54%] | 0.0027 |
| IV→150 mg (N = 38) | 32 | 2.70 ± 3.801 | 1.58 ± 3.869 | −1.13 ± 1.675 [42%] | 0.0790 |
| Placebo (N = 33) | 28 | 5.73 ± 9.748 | 5.07 ± 8.600 | −0.66 ± 2.553 [12%] | |

Berlin spine score

| | | | | | |
|---|---|---|---|---|---|
| IV→75 mg (N = 34) | 30 | 5.02 ± 7.580 | 2.48 ± 5.410 | −2.53 ± 4.096 [50%] | 0.0063 |
| IV→150 mg (N = 38) | 32 | 2.23 ± 2.826 | 1.16 ± 2.474 | −1.08 ± 1.403 [48%] | 0.0570 |
| Placebo (N = 33) | 28 | 4.50 ± 7.617 | 3.95 ± 6.820 | −0.55 ± 2.447 [12%] | |

MRI Subset: a subgroup of patients who have MRI performed at selected centers.
[%] = Mean Change/Mean Base × 100%

TABLE 7
MRI measurements at baseline, Week 52 and change from baseline
(MRI subset of TNF-alpha inhibitor naive patients)

| | | | | |
|---|---|---|---|---|
| | | Baseline | Week 52 | Change from baseline |
| MRI variable | n | (mean ± SD) | (mean ± SD) | (mean ± SD) |

Berlin sacroiliac joint total edema score

| | | | | |
|---|---|---|---|---|
| IV→75 mg | 27 | 2.22 ± 3.283 | 0.76 ± 1.077 | −1.46 ± 2.631 [66%] |
| (N = 34) | | | | |
| IV→150 mg | 32 | 2.22 ± 3.377 | 0.91 ± 1.706 | −1.31 ± 2.317 [59%] |
| (N = 38) | | | | |

Total ASspi-MRI-a score

| | | | | |
|---|---|---|---|---|
| IV→75 mg | 27 | 6.85 ± 11.229 | 2.70 ± 6.445 | −4.15 ± 7.618 [61%] |
| (N = 34) | | | | |
| IV→150 mg | 32 | 2.47 ± 3.726 | 1.63 ± 4.143 | −0.84 ± 2.418 [34%] |
| (N = 38) | | | | |

Berlin spine score

| | | | | |
|---|---|---|---|---|
| IV→75 mg | 27 | 5.41 ± 7.887 | 2.20 ± 5.128 | −3.20 ± 5.131 [59%] |
| (N = 34) | | | | |
| IV→150 mg | 32 | 2.09 ± 2.821 | 1.20 ± 2.599 | −0.89 ± 1.754 [43%] |
| (N = 38) | | | | |

MRI Subset: a subgroup of patients who have MRI performed at selected centers.
[%] = Mean Change/Mean Base × 100%

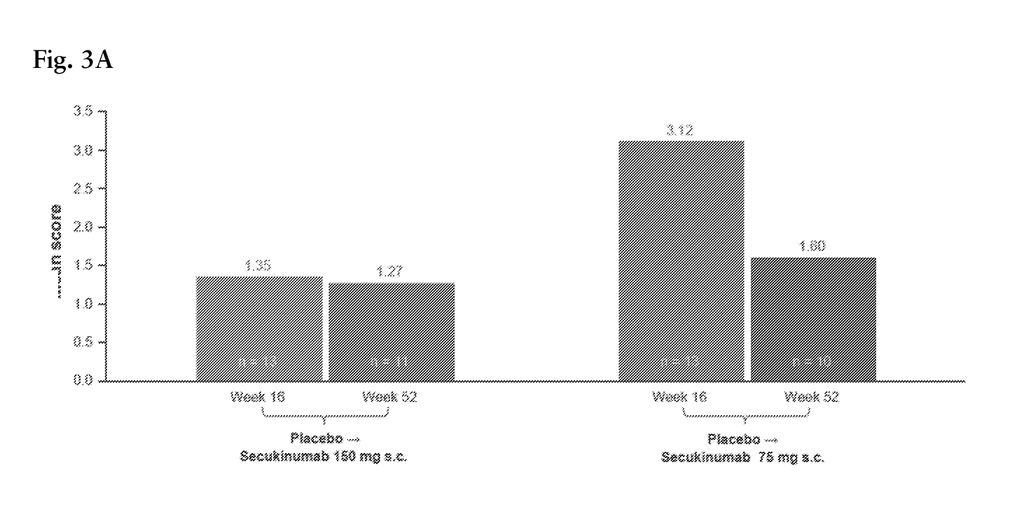

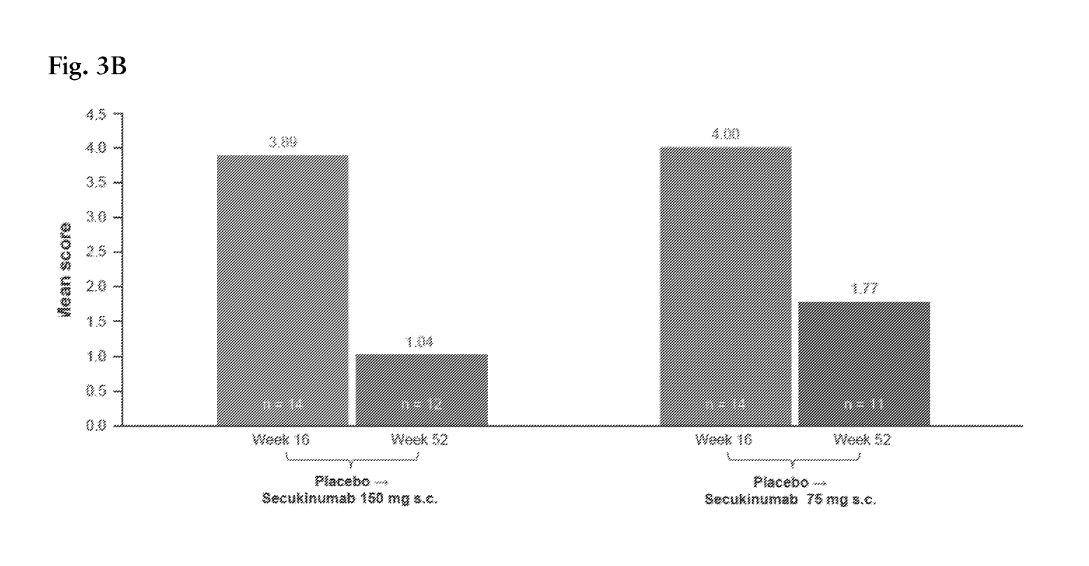

MRI measures demonstrate that secukinumab provides early reductions in spinal inflammation in subjects with active AS, with improvements sustained through 52 weeks of therapy. Subjects who were switched from placebo to monthly SC secukinumab at Weeks 16 and 24 showed an improvement in the Berlin SI joint total oedema score (FIG. 3A) and the Berlin spine score at Week 52 from the respective Week 16 scores (FIG. 3B).

### Example 2.3—Imaging Analysis CAIN457F2305 at Week 104

X-rays of the cervical, thoracic and lumbar spine were performed at baseline and Week 104. A summary of mSASSS and RASSS scores and change from baseline for the originally randomized secukinumab dose groups and for placebo patients who switched to secukinumab treatment is shown in Table 8, with increases in mSASSS and RASSS scores indicating worsening structural progression. Only patients with paired X-ray data at both baseline and Week 104 were analyzed. The placebo-secukinumab groups in these analyses pooled both placebo non-responders and responders re-randomized to secukinumab.

In the overall population of patients randomized to secukinumab at study start, the mean change from baseline in mSASSS at Week 104 was 0.30 for the IV→150 mg group and 0.31 for the IV→75 mg group. Similar changes from baseline were observed in anti-TNF-α naïve patients (0.37 for IV→0.150 mg and 0.36 for IV→75 mg) but were lower in TNF-IR patients (0.14 and 0.13, respectively). The change from baseline in RASSS at Week 104 showed results consistent with the Week 104 mSASSS data.

For placebo patients who switched to secukinumab SC dosing at Week 16 (non-responder) or Week 24 (responder) and therefore had 4-6 months less exposure to secukinumab with no iv loading regimen, there was a slightly greater increase from baseline in mSASSS (0.44 for placebo→150 mg and 0.64 for placebo→75 mg) relative to patients treated with secukinumab from study start. This pattern was observed in both anti-TNF-α naïve patients and TNF-IR patients. Similar results as for mSASSS were observed in RASSS change from baseline at 2 years.

TABLE 8
mSASSS and RASSS at baseline and 2 years (FAS)

| | mSASSS | | | RASSS | | |
|---|---|---|---|---|---|---|
| | Baseline | Week 104 | Change | Baseline | Week 104 | Change |
| | mean | mean | mean | mean | mean | mean |
| Variable | (SD) | (SD) | (SD) | (SD) | (SD) | (SD) |

Overall population

| | | | | | | |
|---|---|---|---|---|---|---|
| IV→75 mg (n = 82) | 10.84 | 11.15 | 0.31 | 12.87 | 13.15 | 0.28 |
| | (16.693) | (16.488) | (3.037) | (19.666) | (19.315) | (3.370) |
| IV→150 mg (n = 86) | 9.63 | 9.92 | 0.30 | 10.86 | 11.30 | 0.45 |
| | (16.632) | (16.867) | (1.935) | (19.219) | (19.685) | (2.082) |
| Placebo→75 mg | 10.59 | 11.23 | 0.64 | 12.65 | 13.35 | 0.71 |
| (n = 44) | (16.320) | (17.151) | (2.788) | (18.788) | (19.698) | (2.798) |
| Placebo→150 mg | 9.60 | 10.04 | 0.44 | 11.34 | 11.76 | 0.41 |
| (n = 45) | (16.097) | (16.754) | (2.092) | (18.795) | (19.442) | (2.188) |

anti-TNF-α naïve patients

| | | | | | | |
|---|---|---|---|---|---|---|
| IV→75 mg (n = 62) | 10.36 | 10.73 | 0.36 | 12.39 | 12.73 | 0.34 |
| | (16.624) | (16.318) | (2.823) | (19.674) | (19.234) | (3.385) |
| IV→150 mg (n = 60) | 9.19 | 9.56 | 0.37 | 9.98 | 10.55 | 0.58 |
| | (16.142) | (16.142) | (2.257) | (18.304) | (18.908) | (2.358) |
| Placebo→75 mg | 8.78 | 9.28 | 0.50 | 10.47 | 11.06 | 0.59 |
| (n = 32) | (16.089) | (17.148) | (3.173) | (18.716) | (19.853) | (3.189) |
| Placebo→150 mg | 10.93 | 11.38 | 0.46 | 12.47 | 12.82 | 0.35 |
| (n = 34) | (17.250) | (18.000) | (2.359) | (19.812) | (20.508) | (2.445) |

TNF-IR patients

| | | | | | | |
|---|---|---|---|---|---|---|
| IV→75 mg (n = 20) | 12.33 | 12.45 | 0.13 | 14.35 | 14.45 | 0.10 |
| | (17.255) | (17.370) | (3.699) | (20.076) | (20.008) | (3.405) |
| IV→150 mg (n = 24) | 10.64 | 10.77 | 0.14 | 12.89 | 13.04 | 0.15 |
| | (18.001) | (18.194) | (0.819) | (21.424) | (21.663) | (1.223) |
| Placebo→75 mg | 15.42 | 16.42 | 1.00 | 18.46 | 19.46 | 1.00 |
| (n = 12) | (16.636) | (16.759) | (1.348) | (18.495) | (18.710) | (1.348) |
| Placebo→150 mg | 5.50 | 5.91 | 0.41 | 7.86 | 8.46 | 0.59 |
| (n = 11) | (11.563) | (11.870) | (0.944) | (15.536) | (16.107) | (1.136) |

n = number of patients with paired X-ray data at both baseline and Week 104;
SD = standard deviation
Maximum total score is 72 for mSASSS and 84 for RASSS

Probability plots of radiographic progression were generated (data not shown). Approximately 80% of patients in the IV→150 mg and IV→75 mg groups showed no radiographic progression (change from baseline ≤0) according to mSASSS and RASSS scores over 2 years of treatment with secukinumab. These high rates were observed in both TNF-IR and anti-TNF-α naïve patients in the IV→150 mg and IV→75 mg dose groups. Similarly high rates of non-progression were also observed in placebo →150 mg SC or placebo →75 mg SC without an IV loading regimen.

### Example 3: Phase III Clinical Trial CAIN457F2310 (MEASURE 2)

MEASURE 2 (NCT01649375) is a randomized, double-blind, placebo (PBO)-controlled, phase 3 trial, which has previously shown that subcutaneous (SC) administration of the human anti-IL-17A monoclonal antibody secukinumab rapidly reduces the signs and symptoms of ankylosing spondylitis (AS) through 16 weeks of therapy.

### Example 3.1—Results CAIN457F2310

Here, our goal is to investigate the long-term efficacy and safety of SC secukinumab in subjects enrolled in the MEASURE 2. 219 adults with active AS, despite maximally tolerated therapy with nonsteroidal anti-inflammatory drugs (NSAIDs), were randomized to receive SC secukinumab 150 mg, 75 mg, or PBO at baseline, Weeks 1, 2, 3 and 4, and every 4 weeks thereafter. At Week 16, subjects in the PBO group were re-randomized to secukinumab 150 mg or 75 mg every 4 weeks. The primary endpoint was the proportion of subjects achieving an Assessment of Spondyloarthritis International Society (ASAS) 20 response at Week 16. Secondary endpoints included ASAS40, high sensitivity C-reactive protein (hsCRP), ASAS 5/6, Bath Ankylosing Spondylitis Disease Activity (BASDAI), Short Form-36 Health Survey Physical Component Summary (SF-36 PCS), Ankylosing Spondylitis Quality of Life (ASQoL), and ASAS partial remission. Statistical analyses at Week 16 used non-responder imputation (binary variables) and mixed-effects repeated measures model (continuous variables), following a pre-defined hierarchical hypothesis testing strategy to adjust for multiplicity of testing. Week 52 data are presented as observed.

181 pts (82.6%) completed 52 weeks of treatment. ASAS20 response rate at Week 16 was 61.1% with secukinumab 150 mg vs 28.4% with PBO (P=0.0001) (Table 9). Secukinumab 150 mg also significantly improved hsCRP, ASAS40, ASAS 5/6, BASDAI, SF-36 PCS and ASQoL at Week 16, compared with PBO. Clinical responses with secukinumab 75 mg did not reach statistical significance for any of the pre-specified endpoints based on hierarchical testing. Improvements with secukinumab 150 mg were sustained through Week 52; ASAS20/40 response rates with secukinumab 150 mg were 73.8%/57.4% at Week 52 (observed data). Over the entire treatment period (mean secukinumab exposure: 425.8 days; mean PBO exposure: 107.6 days), exposure-adjusted adverse event (AE) rates were 214.1, 211.7 and 443.2 per 100 patient-years amongst secukinumab 150 mg-, 75 mg- and PBO-treated subjects, respectively.

TABLE 9
Primary and Secondary Endpoint Results at Weeks 16 and 52.

| | | Secukinumab | Secukinumab | |
|---|---|---|---|---|
| | | 150 mg SC | 75 mg SC | Placebo |
| ASAS20, % | Wk 16 | 61.1^{†} | 41.1 | 28.4 |
| | Wk 52 | 73.8 | 63.9 | N/A |
| ASAS40, % | Wk 16 | 36.1^{†} | 26.0 | 10.8 |
| | Wk 52 | 57.4 | 41.0 | N/A |
| hsCRP, post- | Wk 16 | 0.55^{†} | 0.61 | 1.13 |
| baseline/baseline | Wk 52 | 0.46 | 0.58 | N/A |
| ratio | | | | |
| ASAS 5/6, % | Wk 16 | 43.1^{†} | 34.2 | 8.1 |
| | Wk 52 | 62.3 | 47.5 | N/A |
| BASDAI, mean | Wk 16 | −2.19^{†} | −1.92 | −0.85 |
| change from | Wk 52 | −3.14 | −2.63 | N/A |
| baseline | | | | |
| SF-36 PCS, mean | Wk 16 | 6.06^{†} | 4.77 | 1.92 |
| change from | Wk 52 | 7.99 | 6.62 | N/A |
| baseline | | | | |
| ASQoL, mean | Wk 16 | −4.00^{§} | −3.33 | −1.37 |
| change from | Wk 52 | −5.25 | −4.13 | N/A |
| baseline | | | | |
| ASAS partial | Wk 16 | 13.9 | 15.1 | 4.1 |
| remission, % | Wk 52 | 26.2 | 18.0 | N/A |

^{†}P < 0.001
^{§}P < 0.01 for comparisons vs PBO.
P-values at Week 16 are adjusted for multiplicity.
At Week 16: N = 72 secukinumab 150 mg, N = 73 secukinumab 75 mg, N = 74 placebo;
At Week 52: N = 61 for both secukinumab 150 mg and 75 mg (except for SF-36 PCS where N = 62 and N = 58, respectively, and for ASQoL where N = 60 for secukinumab 75 mg).
NRI (binary variables) and MMRM (continuous variables) data presented at Week 16.
Week 52 data are as observed, except hs-CRP where the post-baseline-to-baseline ratio is presented.
N/A, not applicable.

Secukinumab 150 mg SC rapidly improved the signs and symptoms of disease, reduced inflammation, and improved physical function and health-related quality of life in subjects with AS. Benefits were sustained through 52 weeks of therapy. Secukinumab was well tolerated; safety findings were consistent with previous reports.

### Example 3.3—Efficacy Data by Anti-TNF Alpha Status in CAIN457F2310

In Example 3.2, our goal is to evaluate the efficacy and safety of secukinumab by anti-TNF response status at Weeks 16 and 52 in the MEASURE 2 study.

219 adults with active AS were randomized to receive subcutaneous (SCSC) secukinumab (150 or 75 mg) or PBO at baseline, week 1, 2, 3 and 4, and every 4 weeks thereafter. Randomization was stratified according to prior anti-TNF response status: anti-TNF-naïve or inadequate response or intolerance to not more than one anti-TNF biologic agent (anti-TNF-IR). At week 16 PBO-treated subjects were re-randomized to secukinumab 150 or 75 mg. Preplanned subgroup analyses of the primary and secondary endpoints were conducted among the anti-TNF-naïve and anti-TNF-IR subjects and included: the proportion of subjects achieving an Assessment of SpondyloArthritis International Society (ASAS) 20 response (primary endpoint), ASAS40, high sensitivity C-reactive protein (hsCRP), ASAS 5/6, Bath Ankylosing Spondylitis Disease Activity Index (BASDAI), Short Form-36 Physical Component Summary (SF-36 PCS), Ankylosing Spondylitis Quality of Life (ASQoL), and ASAS partial remission. Analyses at week 16 used non-responder imputation (binary variables) and mixed-effects repeated measures model (continuous variables). Week 52 data are presented as observed

62% of subjects enrolled were anti-TNF-naïve, and 38% were anti-TNF-IR. At week 16 secukinumab 150 mg (but not 75 mg) improved ASAS20 response rates compared with PBO in both anti-TNF-naïve (68.2% vs 31.1%, respectively; P<0.001) and anti-TNF-IR (50.0% vs 24.1%; P<0.05) subjects. Improvements with secukinumab 150 mg were observed for all secondary endpoints in anti-TNF-naïve subjects, except ASAS partial remission, and for most secondary endpoints in anti-TNF-IR subjects (Table 10). Results for secukinumab 75 mg were in general lower than for secukinumab 150 mg and did not differentiate from placebo for ASAS 20 response rate at Week 16. Clinical responses to secukinumab were sustained or continued to improve in both anti-TNF-naïve and anti-TNF-IR subjects through 52 weeks (Table 10).

TABLE 10
Measures of disease activity and health-related QoL
by anti-TNF experience at Week 16 and Week 52.

| | | Anti-TNF-naïve | | | Anti-TNF-IR | | |
|---|---|---|---|---|---|---|---|
| | | Secukinumab SC | | | Secukinumab SC | | |
| | | 150 mg | 75 mg | PBO | 150 mg | 75 mg | PBO |
| N at Wk 16, Wk 52 | | 44, 39 | 45, 42 | 45, N/A | 28, 22 | 28, 19 | 29, N/A |
| ASAS 20, | Wk 16 | 68.2^{†} | 51.1 | 31.1 | 50.0^{‡} | 25.0 | 24.1 |
| % responders | Wk 52^{a} | 82.1 | 71.4 | N/A | 59.1 | 47.4 | N/A |
| ASAS 40, | Wk 16 | 43.2^{‡} | 31.1 | 17.8 | 25.0^{§} | 17.9^{‡} | 0.0 |
| % responders | Wk 52^{a} | 64.1 | 47.6 | N/A | 45.5 | 26.3 | N/A |
| hsCRP, | Wk 16 | 0.46* | 0.49* | 1.00 | 0.69^{‡} | 0.84 | 1.27 |
| post-baseline/ | Wk 52^{a} | 0.23 | 0.42 | N/A | 0.27 | 0.36 | N/A |
| baseline ratio | | | | | | | |
| ASAS 5/6, | Wk 16 | 50.0^{†} | 40.0^{§} | 13.3 | 32.1^{§} | 25.0^{§} | 0.0 |
| % responders | Wk 52^{a} | 71.8 | 54.8 | N/A | 45.5 | 33.3^{b} | N/A |
| BASDAI, | Wk 16 | −2.56^{§} | −2.27^{‡} | −1.15 | −1.60 | −1.38 | −0.59 |
| mean change from | Wk 52^{a} | −3.33 | −2.86 | N/A | −2.80 | −2.12 | N/A |
| baseline | | | | | | | |
| SF-36 PCS, | Wk 16 | 7.46^{§} | 5.95^{‡} | 2.96 | 4.49^{‡} | 3.57 | 0.34 |
| mean change from | Wk 52^{a} | 8.44^{c} | 7.16^{d} | N/A | 7.18 | 5.33^{e} | N/A |
| baseline | | | | | | | |
| ASQoL, | Wk 16 | −5.02^{§} | −3.97^{‡} | −1.94 | −2.39 | −2.53 | −0.49 |
| mean change from | Wk 52^{a} | −6.04^{c} | −4.19 | N/A | −3.67^{f} | −4.00^{b} | N/A |
| baseline | | | | | | | |
| ASAS partial | Wk 16 | 18.2 | 20.0 | 6.7 | 7.1 | 7.1 | 0 |
| remission, | Wk 52^{a} | 30.8 | 21.4 | N/A | 18.2 | 10.5 | N/A |
| % responders | | | | | | | |

*P < 0.0001,
^{†}P < 0.001,
^{§}P < 0.01,
^{‡}P < 0.05 vs PBO;
^{a}Observed data at Wk 52;
^{b}N = 18;
^{c}N = 40;
^{d}N = 41;
^{e}N = 17;
^{f}N = 21.
N/A, not applicable.

Secukinumab 150 mg SCSC improved the signs and symptoms of AS, reduced inflammation and improved physical function and health-related QoL in both anti-TNF-naïve and anti-TNF-IR subjects.

### Example 4: Comparison of Secukinumab to TNF-Alpha Inhibitors in the Treatment of Nr-axSpA

In radiographic axial spondyloarthritis (AS) patients, secukinumab showed comparable efficacy to TNF alpha inhibitors. In a network meta-analysis comparing data from clinical trials in AS between secukinumab 150 mg SC and all approved TNF alpha inhibitors performed by RTI health solutions, no significant differences were observed for the efficacy endpoints (ASAS20, ASAS40, BASDAI50, ASAS PR, ASAS 5/6, BASFI change from baseline) using pair-wise comparisons (data not shown).

Studies with TNF alpha inhibitors have shown that response to treatment is very similar in patients with active radiographic AS and patients with active nr-axSpA. Specifically, in the RAPID-axSpA trial of certolizumab pegol (Cimzia), both, patients with AS and nr-axSpA were enrolled (Landewé R et al. (2014) Ann Rheum Dis 2014; 73:39-47). The inclusion criteria for patients with nr-axSpA were very similar to the ones for the secukinumab trial in nr-axSpA CAIN457H2315 outlined in Example 5, including active disease defined by BASDAI≥4, spinal pain ≥4, and CRP>ULN and/or SJI MRI. Furthermore, patients had to have an inadequate response or intolerance to NSAIDs.

In the RAPID-axSpA trial, the primary endpoint ASAS20 response was achieved by 56.9% (CZP 200 mg Q2W) to 64.3% (CZP 400 mg Q4W) of AS patients and by 58.7% (CZP 200 mg Q2W) to 62.7% (CZP 400 mg Q4W) of nr-axSpA patients. The secondary endpoint ASAS40 response was achieved by 40.0% (CZP 200 mg Q2W) to 50.0% (CZP 400 mg Q4W) of AS patients and by 47.1% (CZP 400 mg Q4W) to 47.8% (CZP 200 mg Q2W) of nr-axSpA patients. Consistently, also for other endpoints including ASAS PR, ASAS 5/6 and BASDAI mean change from baseline very similar efficacy of certolizumab pegol in both AS and nr-axSpA patients was observed.

In addition to the direct comparison of nr-axSpA and AS patients in the RAPID-axSpA trial, also indirect comparisons between efficacy in nr-axSpA and AS patients for adalimumab show very similar response rates in both patient groups. Here, ASAS20 response was achieved by 58.2% of AS patients (van der Heijde et al (2006) Arthritis Rheum 2006; 54(7):2136-46) and by 51.6% of nr-axSpA patients (Sieper et al (2013) Ann Rheum Dis 2013; 72:815-822). Consistently, ASAS40 was achieved by 39.9% of AS patients (van der Heijde et al (2006) Arthritis Rheum 2006; 54(7):2136-46) and by 36.3% of nr-axSpA patients (Sieper et al (2013) Ann Rheum Dis 2013; 72:815-822).

Based on the evidence above for very similar response rates to TNF alpha inhibitors in nr-axSpA and AS patients, and the very similar efficacy of secukinumab and TNF alpha inhibitors in patient with AS, it is expected that secukinumab will be effective in the treatment of patients with nr-axSpA.

### Example 5: Clinical Trial CAIN457H2315

### Example 5.1: Purpose and Study Objectives

The purpose of this study is to demonstrate the clinical efficacy, safety and tolerability of secukinumab compared with placebo in patients with nr-axSpA at week 16 as well as week 52. Additionally, 1 year progression of structural changes as evidenced by MRI will be assessed at week 52. This study will also observe the long-term efficacy, safety, tolerability of secukinumab and the evolution of radiographic correlates of inflammation and structural progression based on the MRI and X-ray results up to week 104. The primary objective is to demonstrate superiority of secukinumab 150 mg SC over placebo at Week 16 (for the EMA) or Week 52 (for the FDA) in the proportion of subjects achieving an ASAS 40 response (Assessment of SpondyloArthritis International Society criteria). Secondary objectives include demonstrating that the efficacy of secukinumab 150 mg SC at week 16 and week 52 is superior to placebo based on the following: the proportion of patients meeting the ASAS 5/6 response criteria, the change from baseline in total Bath Ankylosing Spondylitis Disease Activity Index (BASDAI), the proportion of subjects achieving BASDAI 50, the change from baseline in Short Form-36 Physical Component Summary (SF-36 PCS), the proportion of subjects achieving an ASAS 20 response, the change from baseline in total Bath Ankylosing Spondylitis Functional Index (BASFI), the change from screening in SI joint edema on MRI, the proportion of patients achieving ASAS partial remission, the proportion of patients achieving Ankylosing Spondylitis Disease Activity Score (ASDAS)—C-Reactive Protein (CRP) inactive disease as defined by ASDAS<1.3, and the proportion of subjects achieving an ASAS40 response at Week 16.

### Example 5.2: Study Design

Study H2315 is a randomized, double-blind, placebo-controlled study. Approximately 555 patients will be randomized to one of three treatment groups (secukinumab 150 mg with SC loading, secukinumab 150 mg without SC loading, or placebo in a ratio of 1:1:1):

Group 1 (secukinumab 150 mg load): secukinumab 150 mg (1 mL, 150 mg/mL) SC prefilled syringe (PFS) at BSL, Weeks 1, 2 and 3, followed by administration every four weeks starting at Week 4;

Group 2 (secukinumab 150 mg No Load): secukinumab 150 mg (1 mL, 150 mg/mL) SC PFS at BSL, placebo at Weeks 1, 2 and 3, followed by secukinumab 150 mg PFS administration every four weeks starting at Week 4;

Group 3 (placebo): placebo (1 mL) SC PFS at BSL, weeks 1, 2, 3, followed by administration every four weeks starting at Week 4.

Based on the clinical judgment of disease activity by the investigator and the patient, background medications, such as NSAIDs and DMARDs, may be modified or added to treat signs and symptoms of nr-axSpA from Week 16 on. Furthermore, patients who are repeatedly (e.g. at two or more consecutive visits) considered to be inadequate responders based on the clinical judgment of disease activity by the investigator and the patient, may receive secukinumab 150 mg s.c. or other biologics as standard of care treatment from Week 20 on. Patients will be stratified at randomization according to the subgroup of objective signs of inflammation they belong to (based on their CRP and MRI status at screening). The only condition that will be placed on enrollment is that no less than 15% of patients should belong to either of the three subgroups of objective signs of inflammation: CRP+ and MRI+, CRP+ and MRI−, CRP− and MRI+.

Additionally, it is planned to enroll no more than approximately 30% TNF-IR patients in the study. Starting at Week 52, all patients will be assigned to receive secukinumab 150 mg s.c. in an open label fashion except for those patients who discontinued blinded study treatment (secukinumab 150 mg or placebo) during the initial 52 weeks of the study.

The originally randomized treatment assignment (secukinumab 150 mg or placebo) will remain blinded until all patients have completed the Week 52 visit. After all patients have completed the Treatment Period 2 (Week 52) and the Week 52 database lock has occurred, site personnel and patients may be unblinded to the original randomized treatment assignment at baseline. All patients will continue to receive secukinumab as open-label treatment up to Week 100, unless they have discontinued study treatment.

A follow-up visit is to be done 12 weeks after last administration of study treatment for all patients, regardless of whether they complete the entire study as planned or discontinue prematurely.

Subjects who complete the 2 year trial may be eligible to enter a planned extension study. The dosing regimen in this study is based upon two phase III trials (CAIN457F2305, CAIN457F2310) in AS. The Phase III trials in AS, CAIN457F2305 and CAIN457F2310, assessed the efficacy of both 75 mg and 150 mg SC maintenance doses with loading regimens consisting of either intravenous doses (CAIN457F2305: 3 doses of 10 mg/kg IV, given every 2 weeks at BSL, weeks 2 and 4) or subcutaneous doses (CAIN457F2310: 4 weekly SC doses matching the maintenance dose of either 75 mg or 150 mg SC given at BSL, weeks 1, 2, and 3). Given the similarity of the ASAS20 and ASAS40 response rates, respectively, at the Week 16 primary endpoint for the 150 mg dose in each of these studies, regardless of whether the loading dosing was IV (CAIN457F2305: 60.8% for IV→150 mg vs 28.7% for placebo for ASAS20 and 41.6% for IV→150 mg vs 13.1% for placebo for ASAS40) or SC (CAIN457F2310: 61.1% for 150 mg SC vs 27.0% for placebo for ASAS20 and 36.1% for 150 mg SC vs 10.8% for placebo for ASAS40), 150 mg SC is a sufficient dose to provide clinically and statistically significant efficacy, whereas higher secukinumab exposures do not appear to confer greater efficacy in AS.

In addition to evaluating the 150 mg SC loading and maintenance regimen for efficacy compared with placebo, this study will also assess the impact of the SC loading regimen itself on efficacy by including a treatment arm of 150 mg maintenance dosing without a SC loading regimen. Thus, the loading regimen (150 mg Load) will assess initial weekly administration of 150 mg for 4 weeks (BSL, Weeks 1, 2, and 3) followed by maintenance dosing every 4 weeks at the same dose starting at Week 4, whereas the No Load regimen will assess dosing of 150 mg given every 4 weeks from BSL onward, with placebo dosing given during the loading phase to mask the two active treatment regimens. Both secukinumab regimens will be compared to a placebo arm whose dosing simulates the loading regimen, in order to blind placebo treatment compared to either active treatment arm.

### Example 5.3: Inclusion and Exclusion Criteria

Patients eligible for inclusion in this study have to fulfill all of the following criteria:

1. Patient must be able to understand and communicate with the investigator and comply with the requirements of the study and must give a written, signed and dated informed consent before any study assessment is performed

2. Male or non-pregnant, non-nursing female patients at least 18 years of age

3. Diagnosis of axSpA according to ASAS axSpA criteria:
a. Inflammatory back pain for at least 6 months;
b. Onset before 45 years of age; and
c. Sacroiliitis on MRI with ≥1 SpA feature OR HLA-B-27 positive with ≥2 SpA features

4. Objective signs of inflammation at screening, evident by:
MRI with Sacroiliac Joint inflammation; and/or
hsCRP>ULN (as defined by the central lab);

5. Active axSpA, as assessed by total BASDAI 24 cm (0-10 cm) at baseline.

6. Spinal pain as measured by BASDAI question #2≥4 cm (0-10 cm) at baseline.

7. Total back pain as measured by VAS≥40 mm (0-100 mm) at baseline.

8. Patients should have been on at least 2 different NSAIDs at the highest recommended dose for at least 4 weeks in total prior to randomization with an inadequate response or failure to respond, or less if therapy had to be withdrawn due to intolerance, toxicity or contraindications

9. Patients who are regularly taking NSAIDs (including COX-1 or COX-2 inhibitors) as part of their axSpA therapy are required to be on a stable dose for at least 2 weeks before randomization

10. Patients who have been on a TNFα inhibitor (not more than one) must have experienced an inadequate response to previous or current treatment given at an approved dose for at least 3 months prior to randomization or have been intolerant to at least one administration of an anti-TNFα agent

11. Patients who have previously been on a TNFα inhibitor will be allowed entry into study after an appropriate wash-out period prior to randomization.

12. Patients taking MTX (≤25 mg/week) or sulfasalazine (≤3 g/day) are allowed to continue their medication and must have taken it for at least 3 months and have to be on a stable dose for at least 4 weeks prior to randomization

13. Patients on MTX must be on stable folic acid supplementation before randomization

14. Patients who are on a DMARD other than MTX or sulfasalazine must discontinue the DMARD 4 weeks prior to randomization, except for leflunomide, which has to be discontinued for 8 weeks prior to randomization unless a cholestyramine washout has been performed

15. Patients taking systemic corticosteroids have to be on a stable dose of ≤10 mg/day prednisone or equivalent for at least 2 weeks before randomization.

Patients fulfilling any of the following exclusion criteria are not eligible for inclusion in this study. No additional exclusions may be applied by the investigator, in order to ensure that the study population will be representative of all eligible patients.

1. Patients with radiographic evidence for sacroiliitis, grade ≥2 bilaterally or grade ≥3 unilaterally (radiological criterion according to the modified New York diagnostic criteria for AS) as assessed by central reader

2. Inability or unwillingness to undergo MRI (e.g patients with pacemakers, aneurysm clips or metal fragments/foreign objects in the eyes, skin or body that are not MRI compatible)

3. Chest X-ray or MRI with evidence of ongoing infectious or malignant process, obtained within 3 months of screening and evaluated by a qualified physician

4. Patients taking high potency opioid analgesics (e.g., methadone, hydromorphone morphine)

5. Previous exposure to secukinumab or any other biologic drug directly targeting IL-17 or IL-17 receptor

6. Use of any investigational drug and/or devices within 4 weeks of randomization, or a period of 5 half-lives of the investigational drug, whichever is longer

7. History of hypersensitivity to the study drug or its excipients or to drugs of similar chemical classes

8. Any therapy by intra-articular injections (e.g., corticosteroid) within 4 weeks before randomization

9. Any intramuscular corticosteroid injection within 2 weeks before randomization

10. Patients previously treated with any biological immunomodulating agents, except those targeting TNFα

11. Patients who have taken more than one anti-TNFα agent

12. Previous treatment with any cell-depleting therapies including but not limited to anti-CD20 or investigational agents (e.g., CAMPATH, anti-CD4, anti-CD5, anti-CD3, anti-CD19)

13. Pregnant or nursing (lactating) women, where pregnancy is defined as the state of a female after conception and until the termination of gestation, confirmed by a positive human chorionic gonadotropin (hCG) laboratory test

14. Women of child-bearing potential, defined as all women physiologically capable of becoming pregnant, unless they are using effective methods of contraception during entire study or longer if required by locally approved prescribing information (e.g. 20 weeks in EU).

15. Active ongoing inflammatory diseases other than axSpA that might confound the evaluation of the benefit of secukinumab therapy, including inflammatory bowel disease or uveitis

16. Underlying metabolic, hematologic, renal, hepatic, pulmonary, neurologic, endocrine, cardiac, infectious or gastrointestinal conditions, which in the opinion of the investigator immunocompromises the patient and/or places the patient at unacceptable risk for participation in an immunomodulatory therapy

17. Significant medical problems or diseases, including but not limited to the following: uncontrolled hypertension (≥160/95 mmHg), congestive heart failure [New York Heart Association status of class III or IV], uncontrolled diabetes, or very poor functional status unable to perform self-care

18. History of clinically significant liver disease or liver injury as indicated by abnormal liver function tests such as SGOT (AST), SGPT (ALT), alkaline phosphatase, or serum bilirubin. The Investigator should be guided by the following criteria:
Any single parameter may not exceed 2×upper limit of normal (ULN). A single parameter elevated up to and including 2×ULN should be re-checked once more as soon as possible, and in all cases, at least prior to enrollment/randomization, to rule out lab error.
If the total bilirubin concentration is increased above 2×ULN, total bilirubin should be differentiated into the direct and indirect reacting bilirubin.

19. History of renal trauma, glomerulonephritis, or patients with one kidney only, or a serum creatinine level exceeding 1.5 mg/dL (132.6 μmol/L)

20. Screening total WBC count <3,000/μL, or platelets <100,000/μL or neutrophils 1,500/μL or hemoglobin <8.5 g/dL (85 g/L)

21. Active systemic infections during the last two weeks prior to randomization (exception: common cold)

22. History of ongoing, chronic or recurrent infectious disease or evidence of tuberculosis infection as defined by either a positive purified protein derivative (PPD) skin test (the size of induration will be measured after 48-72 hours, and a positive result is defined as an induration of ≥25 mm or according to local practice/guidelines) or a positive QuantiFERON TB-Gold test. Patients with a positive test may participate in the study if further work up (according to local practice/guidelines) establishes conclusively that the patient has no evidence of active tuberculosis. If presence of latent tuberculosis is established, then treatment according to local country guidelines must have been initiated

23. Known infection with human immunodeficiency virus (HIV), hepatitis B or hepatitis C at screening or randomization

24. History of lymphoproliferative disease or any known malignancy or history of malignancy of any organ system within the past 5 years (except for basal cell carcinoma or actinic keratoses that have been treated with no evidence of recurrence in the past 3 months, carcinoma in situ of the cervix or non-invasive malignant colon polyps that have been removed)

25. Current severe progressive or uncontrolled disease which in the judgment of the clinical investigator renders the patient unsuitable for the trial

26. Inability or unwillingness to undergo repeated venipuncture (e.g., because of poor tolerability or lack of access to veins)

27. Inability or unwillingness to receive injections with PFS

28. Any medical or psychiatric condition which, in the Investigator's opinion, would preclude the participant from adhering to the protocol or completing the study per protocol

29. Donation or loss of 400 mL or more of blood within 8 weeks before dosing

30. History or evidence of ongoing alcohol or drug abuse, within the last six months before randomization

31. Plans for administration of live vaccines during the study period or 6 weeks prior to randomization

### Example 5.4: Treatment Arms

Patients will be assigned to one of the following two treatment arms in a 1:1:1 ratio, with approximately 185 subjects each in the following arms:
Group 1: Secukinumab 150 mg Load
Group 2: Secukinumab 150 mg No Load
Group 3: Placebo

Subjects will receive study treatment at BSL, Weeks 1, 2, 3, and 4 followed by treatment every 4 weeks through Week 100. Patients who are repeatedly (e.g. two or more consecutive visits) considered to be inadequate responders based on the clinical judgement of disease activity from Week 20 on, can receive secukinumab 150 mg SC or standard of care treatment. In case the chosen standard of care is a TNFα inhibitor, a 12 week wash-out period has to be observed.

After Week 52 database lock, all patients will receive secukinumab 150 mg SC in open-label fashion, without a loading regimen for patients switching from placebo, unless they have discontinued study treatment. Blinding to the original treatment assignment will be maintained until the treatment period 2 (Week 52) is completed by all patients. Patients will self-administer all secukinumab and placebo doses at the study site or at home, according to the assessment schedule.

### Example 5.5: Efficacy Measurements

Assessment of SpondyloArthritis International Society criteria (ASAS)
Patient's global assessment of disease activity (VAS)
Patient's assessment of back pain intensity (total back pain or nocturnal back pain) (VAS)
Bath Ankylosing Spondylitis Functional Index (BASFI)
Bath Ankylosing Spondylitis Disease Activity Index (BASDAI)
Spinal mobility assessed by BASMI (Bath Ankylosing Spondylitis Metrology Index)
Maastricht Ankylosing Spondylitis Enthesitis Score (MASES) and expanded enthesis sites
hsCRP and ESR
ASDAS-ESR, ASDAS-CRP and ASDAS response categories
44-tender and swollen joint count
EQ-5D
ASQoL
WPAI-GH
SF-36 (PCS and MCS)
FACIT-Fatigue
MRI of spine and sacroiliac joints
X-ray of the cervical, thoracic and lumbar spine assessed by modified Stoke Ankylosing Spondylitis Spinal Score (mSASSS)
X-ray of the sacroiliac joints

The MRI for each subject will include T1 and STIR sequences of the sagittal spine (cervical, thoracic and lumbar) and oblique coronal of the pelvis including both sacroiliac joints. The X-ray requirements include lateral views of the cervical and thoraco-lumbar spine for mSASSS scoring (bottom ⅓ of C2 through top ⅓ of T1, inclusive) and anteroposterior view of the pelvis including visibility of both sacroiliac joints for modified NY criteria for AS determination.

1-28. (canceled)

29. A method of treating a patient having non-radiographic axial spondyloarthritis (nr-axSpA), comprising administering an effective amount of an Interleukin-17 (IL-17) antibody or antigen-binding fragment thereof to a patient in need thereof, wherein the IL-17 antibody or antigen-binding fragment thereof binds to an epitope of an IL-17 homodimer having two mature IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain, wherein the IL-17 antibody or antigen-binding fragment thereof has a K_{D} of about 100-200 pM, and wherein the IL-17 antibody or antigen-binding fragment thereof has an in vivo half-life of about 4 weeks.

30. The method according to claim 29, wherein the patient has severe nr-axSpA or moderate to severe nr-axSpA.

31. The method according to claim 29, wherein the patient has active nr-axSpA as assessed by:
a) total BASDAI≥4 cm (0-10 cm) at baseline, spinal pain as measured by BASDAI question number 2≥4 cm (0-10 cm) at baseline, and total back pain as measured by VAS≥40 mm (0-100 mm) at baseline; or
b) total BASDAI≥4 cm (0-10 cm) at baseline.

32. The method according to claim 29, wherein the patient has nr-axSpA according to the ASAS axSpA criteria.

33. The method according to claim 29, wherein:
a) the patient has had inflammatory back pain for at least three months prior to treatment with the IL-17 antibody or antigen-binding fragment thereof,
b) the onset of the inflammatory back pain of a) occurred before the patient was 45 years old, and
c) the patient has MRI evidence of sacroiliac joint (SIJ) inflammation and has at least one SpA feature or the patient is HLA-B27 positive and has at least two SpA features.

34. The method according to claim 29, wherein the patient has objective signs of inflammation as indicated by:
a) elevated C-reactive protein (CRP) levels;
b) magnetic resonance imaging (MRI) evidence of SIJ inflammation;
c) MRI evidence of SIJ inflammation determined according to the Berlin SIJ scoring method; and/or
d) MRI evidence of inflammation of the spine.

35. The method according to claim 29, wherein the patient does not satisfy the radiological criterion according to the modified New York diagnostic criteria for ankylosing spondylitis.

36. The method according to claim 29, wherein the patient previously failed to respond to, or had an inadequate response to, treatment with a nonsteroidal anti-inflammatory drug (NSAID) or treatment with a Tumor Necrosis Factor (TNF)-alpha inhibitor (TNF-IR).

37. The method according to claim 29, wherein the patient has not been previously treated with a TNF-alpha inhibitor (TNF-naïve).

38. The method according to claim 29, further comprising administering to the patient cyclosporine, hydroxychloroquine, methotrexate, an NSAID, sulfasalazine, leflunomide, prednisolone, prednisone, or methylprednisolone.

39. The method according to claim 29, comprising administering the patient about 75 mg-about 300 mg of the IL-17 antibody or antigen-binding fragment thereof by subcutaneous injection at weeks 0, 1, 2 and 3, followed by once monthly dosing starting at week 4.

40. The method according to claim 29, comprising monthly administering the patient about 75 mg-about 300 mg of the IL-17 antibody or antigen-binding fragment thereof by subcutaneous injection.

41. The method according to claim 29, wherein the IL-17 antibody or antigen-binding fragment thereof comprises:
i) an immunoglobulin heavy chain variable domain (V_{H}) comprising the amino acid sequence set forth as SEQ ID NO:8;
ii) an immunoglobulin light chain variable domain (V_{L}) comprising the amino acid sequence set forth as SEQ ID NO:10;
iii) an immunoglobulin V_{H} domain comprising the amino acid sequence set forth as SEQ ID NO:8 and an immunoglobulin V_{L} domain comprising the amino acid sequence set forth as SEQ ID NO:10;
iv) an immunoglobulin V_{H} domain comprising the hypervariable regions set forth as SEQ ID NO:1, SEQ ID NO:2, and SEQ ID NO:3;
v) an immunoglobulin V_{L} domain comprising the hypervariable regions set forth as SEQ ID NO:4, SEQ ID NO:5 and SEQ ID NO:6;
vi) an immunoglobulin V_{H} domain comprising the hypervariable regions set forth as SEQ ID NO:11, SEQ ID NO:12 and SEQ ID NO:13;
vii) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO:5 and SEQ ID NO:6;
viii) an immunoglobulin V_{H} domain comprising the hypervariable regions set forth as SEQ ID NO: 11, SEQ ID NO:12 and SEQ ID NO:13 and an immunoglobulin V_{L} domain comprising the hypervariable regions set forth as SEQ ID NO:4, SEQ ID NO:5 and SEQ ID NO:6;
ix) an immunoglobulin light chain comprising the amino acid sequence set forth as SEQ ID NO:14;
x) an immunoglobulin heavy chain comprising the amino acid sequence set forth as SEQ ID NO:15; or
xi) an immunoglobulin light chain comprising the amino acid sequence set forth as SEQ ID NO:14 and an immunoglobulin heavy chain comprising the amino acid sequence set forth as SEQ ID NO:15.

42. The method according to claim 41, wherein the IL-17 antibody or antigen-binding fragment thereof is secukinumab.

43. A method of treating a patient having axial spondyloarthritis (axSpA), comprising administering the patient about 150 mg of secukinumab by subcutaneous injection at weeks 0, 1, 2 and 3, followed by once monthly dosing starting at week 4.

44. The method of claim 43, wherein the patient has severe active axSpA.

45. The method of claim 43, wherein the patient has objective signs of inflammation, as indicated by CRP and/or MRI.

46. The method of claim 43, wherein the patient does not have radiographic evidence of ankylosing spondylitis.

47. The method of claim 43, wherein the patient previously had an inadequate response to, or was intolerant to, treatment with an NSAID and/or treatment with a TNF-alpha inhibitor.

48. The method of claim 43, wherein the patient has not been previously treated with a TNF-alpha antagonist.


SEQUENCE LISTING (15 sequences)

SEQ ID NO: 1 - protein, 5 aa, artificial
  CDR1 = hypervariable region 1 of heavy chain of AIN457
NYWMN

SEQ ID NO: 2 - protein, 17 aa, ARTIFICIAL
  CDR2 = hypervariable region 2 of heavy chain of AIN457
AINQDGSEKYYVGSVKG

SEQ ID NO: 3 - protein, 18 aa, ARTIFICIAL
  CDR3 = hypervariable region 3 of heavy chain of AIN457
DYYDILTDYYIHYWYFDL

SEQ ID NO: 4 - protein, 12 aa, ARTIFICIAL
  CDR1′ = hypervariable region 1 of light chain of AIN457
RASQSVSSSYLA

SEQ ID NO: 5 - protein, 7 aa, ARTIFICIAL
  CDR2′ = hypervariable region 2 of light chain AIN457
GASSRAT

SEQ ID NO: 6 - protein, 9 aa, ARTIFICIAL
  CDR3′ = hypervariable region 3 of light chain AIN457
QQYGSSPCT

SEQ ID NO: 7 - DNA, 381 bp, HOMO SAPIENS
  CDS (1)..(381)
gaggtgcagttggtggagtctgggggaggcttggtccagcctggggggtccctgagactctcctgtgcagcctctggattcacctttagtaactattggatgaactgggtccgccaggctccagggaaagggctggagtgggtggccgccataaaccaagatggaagtgagaaatactatgtgggctctgtgaagggccgattcaccatctccagagacaacgccaagaactcactgtatctgcaaatgaacagcctgagagtcgaggacacggctgtgtattactgtgtgagggactattacgatattttgaccgattattacatccactattggtacttcgatctctggggccgtggcaccctggtcactgtctcctca

SEQ ID NO: 8 - protein, 127 aa, HOMO SAPIENS [sequence omitted]

SEQ ID NO: 9 - DNA, 327 bp, HOMO SAPIENS
  CDS (1)..(327)
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccgtgcaccttcggccaagggacacgactggagattaaacga

SEQ ID NO: 10 - protein, 109 aa, HOMO SAPIENS
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCTFGQGTRLEIKR

SEQ ID NO: 11 - protein, 10 aa, artificial
  CDR1-x = hypervariable domain x of heavy chain of AIN457
GFTFSNYWMN

SEQ ID NO: 12 - protein, 11 aa, artificial
  CDR2-x = hypervariable domain of heavy chain x of AIN457
AINQDGSEKYY

SEQ ID NO: 13 - protein, 23 aa, ARTIFICIAL
  CDR3-x = hypervariable domain x of heavy chain AIN457
CVRDYYDILTDYYIHYWYFDLWG

SEQ ID NO: 14 - protein, 215 aa, homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 457 aa, homo sapiens
EVQLVESGGGLVQPGGSLRLSCAASGFTFSNYWMNWVRQAPGKGLEWVAAINQDGSEKYYVGSVKGRFTISRDNAKNSLYLQMNSLRVEDTAVYYCVRDYYDILTDYYIHYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK